SEQUENCE LISTING (57 sequences)

SEQ ID NO: 1 - protein, 50 aa, unidentified
MADDAGAAGGPGGPGGPGMGNRGGFRGGFGSGIRGRGRGRGRGRGRGRGA

SEQ ID NO: 2 - protein, 49 aa, unidentified
ADDAGAAGGPGGPGGPGMGNRGGFRGGFGSGIRGRGRGRGRGRGRGRGA

SEQ ID NO: 3 - protein, 48 aa, unidentified
DDAGAAGGPGGPGGPGMGNRGGFRGGFGSGIRGRGRGRGRGRGRGRGA

SEQ ID NO: 4 - protein, 47 aa, unidentified
DAGAAGGPGGPGGPGMGNRGGFRGGFGSGIRGRGRGRGRGRGRGRGA

SEQ ID NO: 5 - protein, 46 aa, unidentified
AGAAGGPGGPGGPGMGNRGGFRGGFGSGIRGRGRGRGRGRGRGRGA

SEQ ID NO: 6 - protein, 45 aa, unidentified
GAAGGPGGPGGPGMGNRGGFRGGFGSGIRGRGRGRGRGRGRGRGA

SEQ ID NO: 7 - protein, 44 aa, unidentified
AAGGPGGPGGPGMGNRGGFRGGFGSGIRGRGRGRGRGRGRGRGA

SEQ ID NO: 8 - protein, 43 aa, unidentified
AGGPGGPGGPGMGNRGGFRGGFGSGIRGRGRGRGRGRGRGRGA

SEQ ID NO: 9 - protein, 42 aa, unidentified
GGPGGPGGPGMGNRGGFRGGFGSGIRGRGRGRGRGRGRGRGA

SEQ ID NO: 10 - protein, 41 aa, unidentified
GPGGPGGPGMGNRGGFRGGFGSGIRGRGRGRGRGRGRGRGA

SEQ ID NO: 11 - protein, 40 aa, unidentified
PGGPGGPGMGNRGGFRGGFGSGIRGRGRGRGRGRGRGRGA

SEQ ID NO: 12 - protein, 39 aa, unidentified
GGPGGPGMGNRGGFRGGFGSGIRGRGRGRGRGRGRGRGA

SEQ ID NO: 13 - protein, 38 aa, unidentified
GPGGPGMGNRGGFRGGFGSGIRGRGRGRGRGRGRGRGA

SEQ ID NO: 14 - protein, 37 aa, unidentified
PGGPGMGNRGGFRGGFGSGIRGRGRGRGRGRGRGRGA

SEQ ID NO: 15 - protein, 36 aa, unidentified
GGPGMGNRGGFRGGFGSGIRGRGRGRGRGRGRGRGA

SEQ ID NO: 16 - protein, 35 aa, unidentified
GPGMGNRGGFRGGFGSGIRGRGRGRGRGRGRGRGA

SEQ ID NO: 17 - protein, 34 aa, unidentified
PGMGNRGGFRGGFGSGIRGRGRGRGRGRGRGRGA

SEQ ID NO: 18 - protein, 33 aa, unidentified
GMGNRGGFRGGFGSGIRGRGRGRGRGRGRGRGA

SEQ ID NO: 19 - protein, 32 aa, unidentified
MGNRGGFRGGFGSGIRGRGRGRGRGRGRGRGA

SEQ ID NO: 20 - protein, 31 aa, unidentified
GNRGGFRGGFGSGIRGRGRGRGRGRGRGRGA

SEQ ID NO: 21 - protein, 50 aa, unidentified
MADDAGAAGGPGGPGGPGMGNRGGFRGGFGSGIRGRGRGRGRGRGRGRGK

SEQ ID NO: 22 - protein, 49 aa, unidentified
ADDAGAAGGPGGPGGPGMGNRGGFRGGFGSGIRGRGRGRGRGRGRGRGK

SEQ ID NO: 23 - protein, 48 aa, unidentified
DDAGAAGGPGGPGGPGMGNRGGFRGGFGSGIRGRGRGRGRGRGRGRGK

SEQ ID NO: 24 - protein, 47 aa, unidentified
DAGAAGGPGGPGGPGMGNRGGFRGGFGSGIRGRGRGRGRGRGRGRGK

SEQ ID NO: 25 - protein, 46 aa, unidentified
AGAAGGPGGPGGPGMGNRGGFRGGFGSGIRGRGRGRGRGRGRGRGK

SEQ ID NO: 26 - protein, 45 aa, unidentified
GAAGGPGGPGGPGMGNRGGFRGGFGSGIRGRGRGRGRGRGRGRGK

SEQ ID NO: 27 - protein, 44 aa, unidentified
AAGGPGGPGGPGMGNRGGFRGGFGSGIRGRGRGRGRGRGRGRGK

SEQ ID NO: 28 - protein, 43 aa, unidentified
AGGPGGPGGPGMGNRGGFRGGFGSGIRGRGRGRGRGRGRGRGK

SEQ ID NO: 29 - protein, 42 aa, unidentified
GGPGGPGGPGMGNRGGFRGGFGSGIRGRGRGRGRGRGRGRGK

SEQ ID NO: 30 - protein, 41 aa, unidentified
GPGGPGGPGMGNRGGFRGGFGSGIRGRGRGRGRGRGRGRGK

SEQ ID NO: 31 - protein, 40 aa, unidentified
PGGPGGPGMGNRGGFRGGFGSGIRGRGRGRGRGRGRGRGK

SEQ ID NO: 32 - protein, 39 aa, unidentified
GGPGGPGMGNRGGFRGGFGSGIRGRGRGRGRGRGRGRGK

SEQ ID NO: 33 - protein, 38 aa, unidentified
GPGGPGMGNRGGFRGGFGSGIRGRGRGRGRGRGRGRGK

SEQ ID NO: 34 - protein, 37 aa, unidentified
PGGPGMGNRGGFRGGFGSGIRGRGRGRGRGRGRGRGK

SEQ ID NO: 35 - protein, 36 aa, unidentified
GGPGMGNRGGFRGGFGSGIRGRGRGRGRGRGRGRGK

SEQ ID NO: 36 - protein, 35 aa, unidentified
GPGMGNRGGFRGGFGSGIRGRGRGRGRGRGRGRGK

SEQ ID NO: 37 - protein, 34 aa, unidentified
PGMGNRGGFRGGFGSGIRGRGRGRGRGRGRGRGK

SEQ ID NO: 38 - protein, 33 aa, unidentified
GMGNRGGFRGGFGSGIRGRGRGRGRGRGRGRGK

SEQ ID NO: 39 - protein, 32 aa, unidentified
MGNRGGFRGGFGSGIRGRGRGRGRGRGRGRGK

SEQ ID NO: 40 - protein, 31 aa, unidentified
GNRGGFRGGFGSGIRGRGRGRGRGRGRGRGK

SEQ ID NO: 41 - protein, 216 aa, unidentified
MADDAGAAGGPGGPGGPGMGNRGGFRGGFGSGIRGRGRGRGRGRGRGRGAAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 42 - protein, 89 aa, unidentified
MADDAGAAGGPGGPGGPGMGNRGGFRGGFGSGIRGRGRGRGRGRGRGRGAHGEGTFTSDLSKQMEEEAVRLFIEWLKNGGPSSGAPPPS

SEQ ID NO: 43 - protein, 89 aa, unidentified
MADDAGAAGGPGGPGGPGMGNRGGFRGGFGSGIRGRGRGRGRGRGRGRGXHGEGTFTSDLSKQMEEEAVRLFIEWLKNGGPSSGAPPPS

SEQ ID NO: 44 - protein, 81 aa, unidentified
MADDAGAAGGPGGPGGPGMGNRGGFRGGFGSGIRGRGRGRGRGRGRGRGAHAEGTFTSDVSSYLEGQAAKEFIAWLVRGRG

SEQ ID NO: 45 - protein, 81 aa, unidentified
MADDAGAAGGPGGPGGPGMGNRGGFRGGFGSGIRGRGRGRGRGRGRGRGXHAEGTFTSDVSSYLEGQAAKEFIAWLVRGRG

SEQ ID NO: 46 - protein, 131 aa, unidentified
MADDAGAAGGPGGPGGPGMGNRGGFRGGFGSGIRGRGRGRGRGRGRGRGAHAEGTFTSDVSSYLEGQAAKEFIAWLVRGRGMADDAGAAGGPGGPGGPGMGNRGGFRGGFGSGIRGRGRGRGRGRGRGRGA

SEQ ID NO: 47 - protein, 86 aa, unidentified
MADDAGAAGGPGGPGGPGMGNRGGFRGGFGSGIRGRGRGRGRGRGRGRGAHDEFERHAEGTFTSDVSSYLEGQAAKEFIAWLVKGR

SEQ ID NO: 48 - protein, 86 aa, unidentified
MADDAGAAGGPGGPGGPGMGNRGGFRGGFGSGIRGRGRGRGRGRGRGRGXHDEFERHAEGTFTSDVSSYLEGQAAKEFIAWLVKGR

SEQ ID NO: 49 - protein, 58 aa, unidentified
MADDAGAAGGPGGPGGPGMGNRGGFRGGFGSGIRGRGRGRGRGRGRGRGAFCFWKTCT

SEQ ID NO: 50 - protein, 58 aa, unidentified
MADDAGAAGGPGGPGGPGMGNRGGFRGGFGSGIRGRGRGRGRGRGRGRGXFCFWKTCT

SEQ ID NO: 51 - protein, 166 aa, unidentified
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 52 - protein, 8 aa, synthetic construct
FCFWKTCT

SEQ ID NO: 53 - protein, 39 aa, unidentified
HGEGTFTSDLSKQMEEEAVRLFIEWLKNGGPSSGAPPPS

SEQ ID NO: 54 - protein, 32 aa, synthetic construct
HAEGTFTSDVSSYLEGQAAKEEFIAWLVRGRG

SEQ ID NO: 55 - protein, 36 aa, UNIDENTIFIED
HDEFERHAEGTFTSDVSSYLEGQAAKEFIAWLVKGR

SEQ ID NO: 56 - protein, 168 aa, unidentified
GAAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 57 - protein, 48 aa, unidentified
MADDAGAAGGPGGPGGPGMGNRGGFRGGFGSGIRGRGRGRGRGRGRGR

(19) United States
(12) Patent Application Publication
(10) Pub. No.: US 2025/0388638 A1
(43) Pub. Date: Dec. 25, 2025

(54) COMPOSITIONS AND METHODS FOR ORAL DELIVERY

(71) Applicant: IMAGINE PHARMA LLC, PITTSBURGH, PA (US)

(72) Inventors: NGOC THAI, PITTSBURGH, PA (US); JONATHAN POLLETT, PITTSBURGH, PA (US)

(73) Assignee: IMAGINE PHARMA LLC, PITTSBURGH, PA (US)

(21) Appl. No.: 18/683,324

(22) PCT Filed: Dec. 9, 2022
(86) PCT No.: PCT/US2022/081277

§ 371 Date: Sep. 9, 2025

Related U.S. Application Data
(60) Provisional application No. 63/288,579, filed on Dec. 11, 2021.

(51) Int. Cl. C07K 14/505; A61K 9/00; A61K 38/00; A61P 3/10; A61P 7/06; C07K 14/605; C07K 14/62

(52) U.S. Cl. CPC C07K 14/505; A61K 9/0053; A61P 3/10; A61P 7/06; C07K 14/605; C07K 14/62; A61K 38/00

(57) ABSTRACT

Compositions and methods for the targeted delivery of therapeutic polypeptides and protein-based therapeutics across the gastrointestinal lining are disclosed. In one aspect, provided is a polypeptide construct comprising (a) a first polypeptide comprising an amino acid sequence that is at least 80% identical to an amino acid sequence selected from any one of SEQ ID NO: 1-40; and (b) a second polypeptide, wherein the second polypeptide is heterologous to the first polypeptide. In one aspect, the heterologous polypeptide is a therapeutic polypeptide.

### CROSS REFERENCE TO RELATED APPLICATIONS

This application claims the benefit of US Provisional Application. No. 63/288,579, filed Dec. 11, 2021, the content of which is incorporated by reference herein it is entirely.

### BACKGROUND

Oral administration of conventional small molecule or low molecular weight drugs has been a well-established practice. Other therapeutic drugs, such as those comprising peptides and proteins, however, are often unstable, have large molecular weights, and/or are polar in nature, and as a result cannot be administered orally for any meaningful therapeutic effect due to poor permeability through biological membranes. When administered orally, many drugs are susceptible to proteolytic degradation in the gastrointestinal tracts and only pass with difficulty into bodily fluids. For this reason, therapeutic polypeptides and proteins have been administered mostly by injection or infusion, which is significantly less convenient, and significantly more expensive and burdensome, than oral administration.

Proteolytic enzymes of both the stomach and intestines may degrade biologics and polypeptide-based therapeutics, rendering them inactive before they can be absorbed into the bloodstream. Any amount of polypeptide that survives proteolytic degradation by proteases of the stomach (typically having acidic pH) will also undergo action by proteases of the small intestine and enzymes secreted by the pancreas (typically having neutral to basic pH). Specific difficulties arising from the oral administration of a polypeptide involve the relatively large size of the molecule, and the charge distribution it carries. This may make it more difficult for a polypeptide to penetrate the mucus along intestinal walls or to cross into the blood.

Oral administration of therapeutic polypeptides has two main challenges that are a) degradation by proteolytic enzymes in the stomach and intestine and b) poor absorption, i.e., poor transport of the polypeptide to the basolateral side of the intestine and release into the blood. Improving oral effectiveness, i.e., increase of the bioavailability of oral biologics and polypeptide-based drugs, is an unmet medical need.

### SUMMARY

Compositions and methods for the targeted delivery of therapeutic polypeptides and protein-based therapeutics across the gastrointestinal lining are disclosed herein.

In one aspect, provided is a polypeptide construct comprising (a) a first polypeptide comprising an amino acid sequence that is at least 80% identical to an amino acid sequence selected from any one of SEQ ID NO: 1-40; and (b) a second polypeptide, wherein the second polypeptide is heterologous to the first polypeptide. In one aspect, the heterologous polypeptide is a therapeutic polypeptide.

In another aspect, the first polypeptide comprises an amino acid sequence that is at least 90% identical to an amino acid sequence selected from any one of SEQ ID NO: 1-40. In another, the first polypeptide comprises an amino acid sequence that is at least 95% identical to an amino acid sequence selected from any one of SEQ ID NO: 1-40.

In another aspect, the first polypeptide comprises an amino acid sequence that is at least 98% identical to an amino acid sequence selected from any one of SEQ ID NO: 1-40. In another, the first polypeptide comprises an amino acid sequence that is at least 99% identical to an amino acid sequence selected from any one of SEQ ID NO: 1-40. In another, the first polypeptide comprises an amino acid sequence that comprises an amino acid sequence selected from any one of SEQ ID NO: 1-40.

In another aspect, a pharmaceutical composition for targeted delivery across the gastrointestinal lining following oral administration to a subject, comprises a therapeutically effective amount of a polypeptide construct comprising a polypeptide with at least 80% sequence identity to one or more of SEQ ID 1-40, and wherein the polypeptide is linked to a heterologous polypeptide.

In another aspect, the composition for targeted delivery across the gastrointestinal lining following oral administration of the composition to a subject further comprises one or more of a pharmaceutically acceptable additive, excipient, stabilizer, permeability enhancer or protease inhibitor.

In another aspect, disclosed herein are polypeptide constructs suitable for the targeted delivery of a heterologous polypeptide across the gastrointestinal lining of a subject.

In another aspect, a targeted delivery system is composed, comprising a heterologous polypeptide, and means for transporting the heterologous polypeptide across the gastrointestinal lining of a subject, wherein the heterologous polypeptide is a therapeutic polypeptide and wherein the means for transporting comprises providing a polypeptide having a sequence identity of at least 80% to a polypeptide according to SEQ ID 1-40, and linking the polypeptide to the heterologous polypeptide.

In another aspect, a polypeptide construct comprises a polypeptide linked to a heterologous polypeptide by a linker, wherein the linker is an amide bond formed between an alkyl modified peptide on the polypeptide and an azide modified peptide on the heterologous polypeptide.

The modular nature of the disclosed compositions and methods for targeted drug delivery provide advantageous means for oral formulations of polypeptide-based therapeutics, otherwise suitable for administration solely by injection or infusion due to their size or molecular complexity.

### DETAILED DESCRIPTION OF THE DRAWINGS

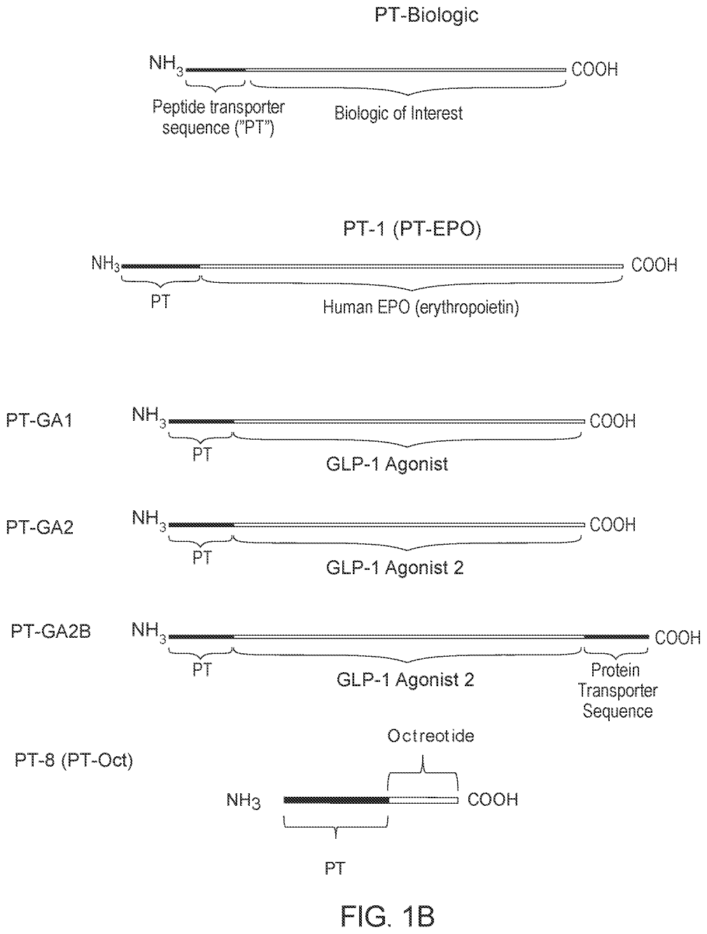

FIG. 1B shows an overview of a drug delivery system that comprises a polypeptide construct comprising (a) a first polypeptide comprising an amino acid sequence that is at least 80% identical to an amino acid sequence selected from any one of SEQ ID NO: 1-40; and (b) a second polypeptide, wherein the second polypeptide is heterologous to the first polypeptide. In one embodiment, the peptide according to SEQ ID 1-40 is linked at the N-terminus or the C-terminus of the therapeutic polypeptide. The therapeutic polypeptide may be a biologic, a peptide-based drug, or a large molecule drug, otherwise not suitable for oral administration. The drug delivery method disclosed provides the means for transforming drug delivering limited to IV/SQ to delivery by PO. Shown in FIG. 1B are representative examples of polypeptide constructs comprising therapeutic polypeptides, for example: Erythropoietin (PT-EPO); GLP-1; GLP-1 Agonist (PT-GA-1; PT-GA2); and Octreotide (PT-OCT), amongst other therapeutic proteins, which are shown in Table 2. (Note: constructs shown in FIG. 1B are not drawn to scale.)

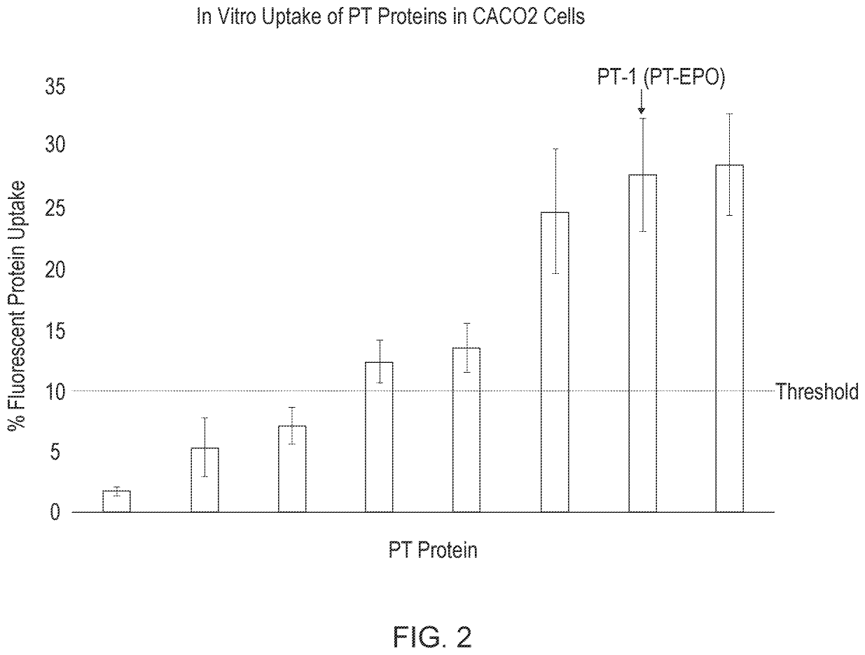

FIG. 2 illustrates the in vivo uptake of polypeptide constructs comprising a polypeptide comprising an amino acid sequence having a sequence identity according to SEQ ID NO: 1-40; and a heterologous polypeptide, as determined by a fluorescence assay in a Caco-2 cell model. (See Example 1) Caco-2 cells may be cultured in the presence of a polypeptide construct comprising a polypeptide and a heterologous polypeptide and analyzed using a fluorescent assay to determine the percentage of uptake of the polypeptide construct into the cells. Polypeptide constructs from left to right are polypeptide constructs comprising a polypeptide according to SEQ ID NO: 1 linked to BSA (bovine serum albumin); linked to Elosulfase alfa; linked to factor VIII; linked to g-csf; linked to belatacept; linked to Glucarpidase; linked to Erythropoietin (EPO); and linked to factor IX. Several peptide constructs were tested for uptake by Caco-2 cells, including various peptide constructs comprising truncated polypeptides. While not shown in the graph, it was determined that truncated polypeptides (as short as 20 amino acids long and represented by the polypeptides according to SEQ ID 1-20) facilitated the uptake of a heterologous polypeptide by Caco-2 cells, compared to control.

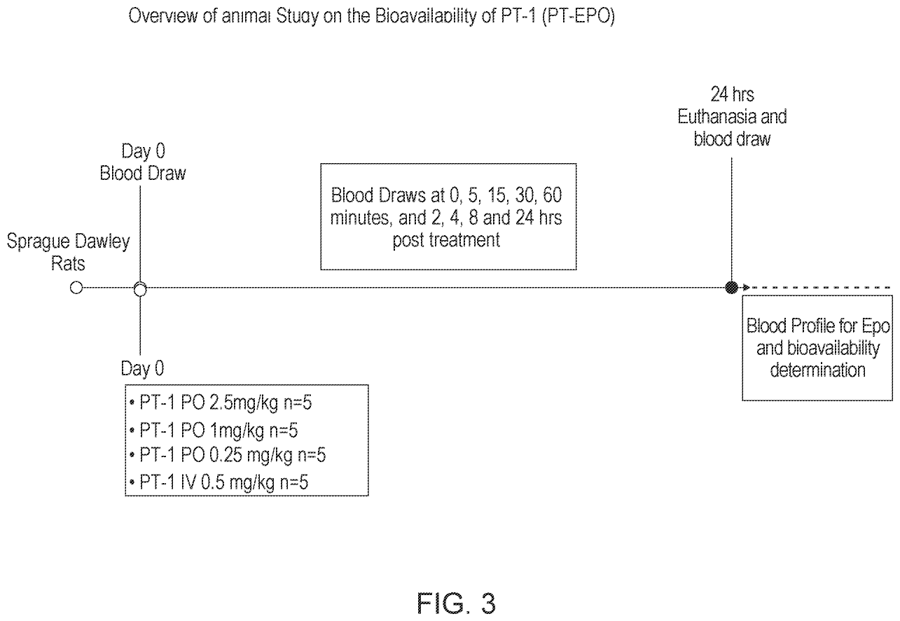

FIG. 3 shows an overview of an in vivo animal study using Sprague Dawley rats to test a targeted delivery method for administration of an oral dose form of human erythropoietin. Sprague Dawley rats (n=8) were administered a composition comprising a polypeptide construct of SEQ ID NO: 41 (referenced in the figure as “PT-EPO”), the polypeptide construct comprising a polypeptide according to SEQ ID NO: 1 and a heterologous polypeptide according to SEQ ID NO: 43. Compositions comprising PT-EPO at concentrations of 2.5 mg/kg, 1 mg/kg, and 0.25 mg/kg in PBS were administered per os (PO). A composition comprising PT-EPO at a concentration of 0.5 mg/kg in PBS was administered intravenously (IV) as a separate control/reference for bioavailability. Blood draws were taken from the treated animals (PO and IV) at time intervals post administration, including: 0, 5, 15, 30, and 60 minutes; and 2, 4, 8 and 24 hours. The samples were tested to assess the presence or absence of human erythropoietin in the bloodstream of the subject animals.

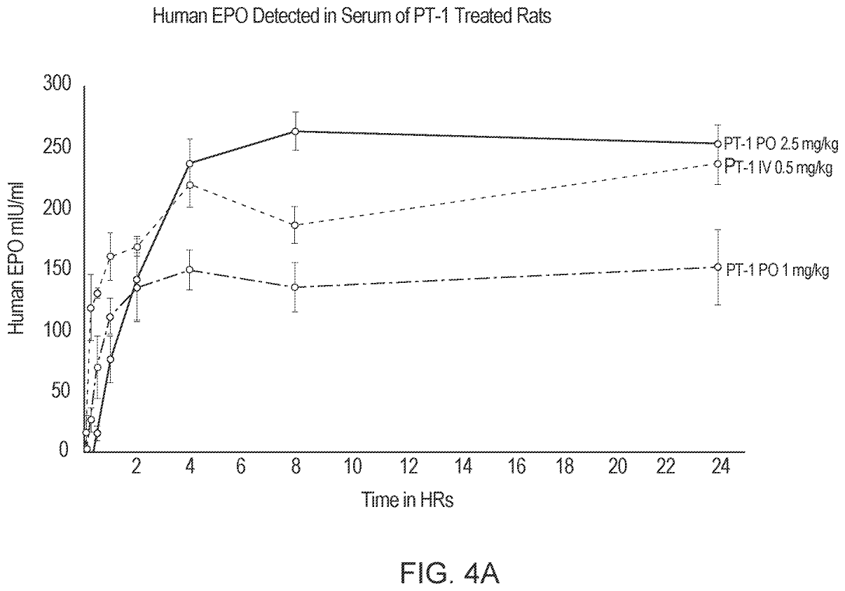

FIG. 4A illustrates the uptake and bioavailability of a targeted delivery method for oral administration of erythropoietin (See Examples 2-5). A composition comprising a polypeptide construct of SEQ ID 41 (PT-EPO) was administered PO and IV, and at three doses. Serum was collected post administration at various time points over a 24-hour period, including: 0, 5, 15, 30, and 60 minutes; and 2, 4, 8 and 24 hours. In all doses administered, a polypeptide according to SEQ ID 56 (human erythropoietin with N-terminal glycine and alanine resides) was detected in serum of treated rats. This data confirms that a polypeptide construct of SEQ ID 41 crossed the intestinal barrier of the gastrointestinal (GI) tract when administered orally and that a polypeptide of SEQ ID 56 was taken up into the bloodstream.

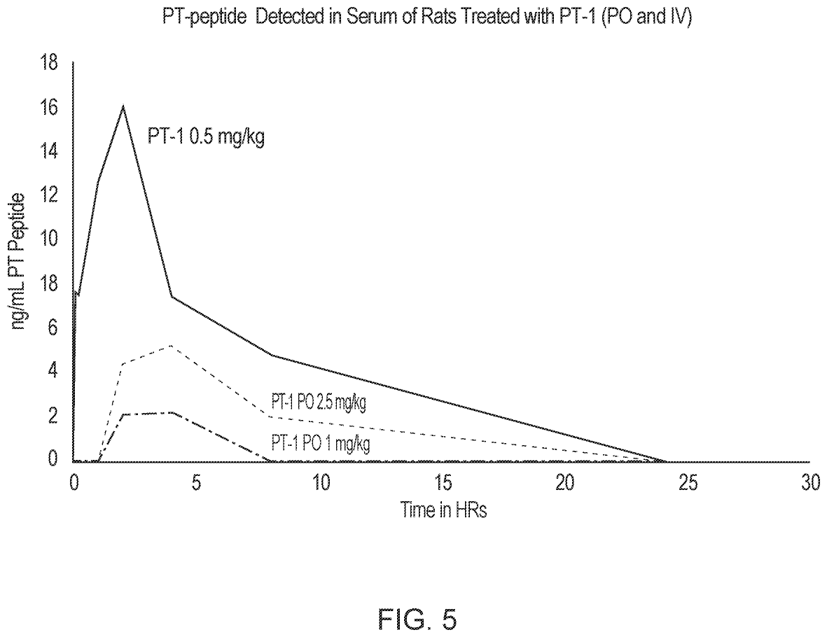

FIG. 5 shows the presence of a polypeptide of SEQ ID 57 in the serum of rats following oral and intravenous administration of a composition comprising the polypeptide construct of SEQ ID 41. The data shows that following administration of the composition, the polypeptide construct is cleaved, resulting in two polypeptide fragments: a polypeptide according to SEQ ID NO: 56 and SEQ ID NO: 57.

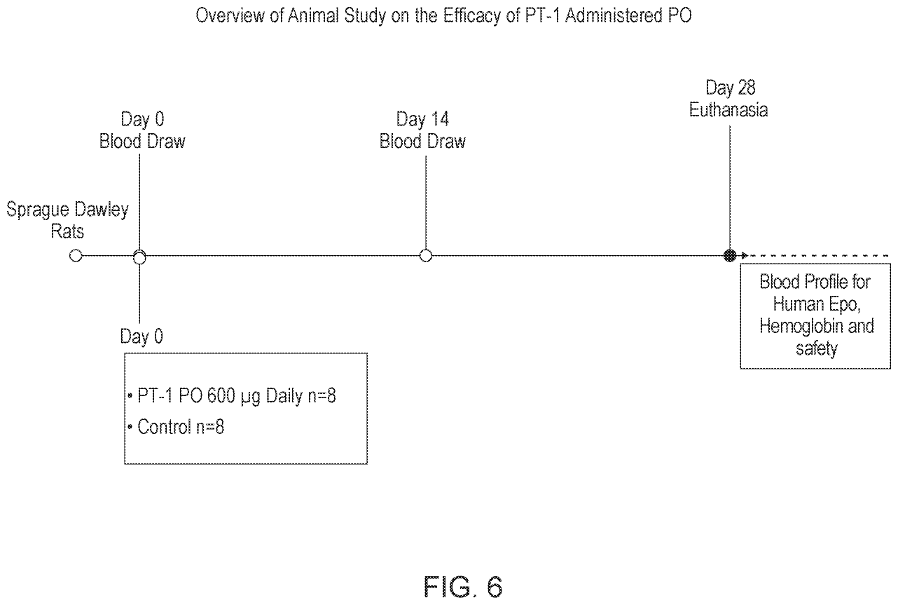

FIG. 6 shows an overview of an animal model study to test the efficacy of a composition comprising a polypeptide according to SEQ ID 41 for targeted delivery of erythropoietin across the gastrointestinal barrier. Sprague Dawley rats (n=8) were administered 600 μg (daily by PO) of a composition comprising a polypeptide construct with a sequence identity corresponding to SEQ ID 41 (See Example 3). A corresponding control group (n=8) were administered a vehicle control (a PBS solution containing 10 mM maltose) PO. Blood samples were collected at days 0, 14 and 28 and tested for the presence of human erythropoietin. Hemoglobin levels were also measured.

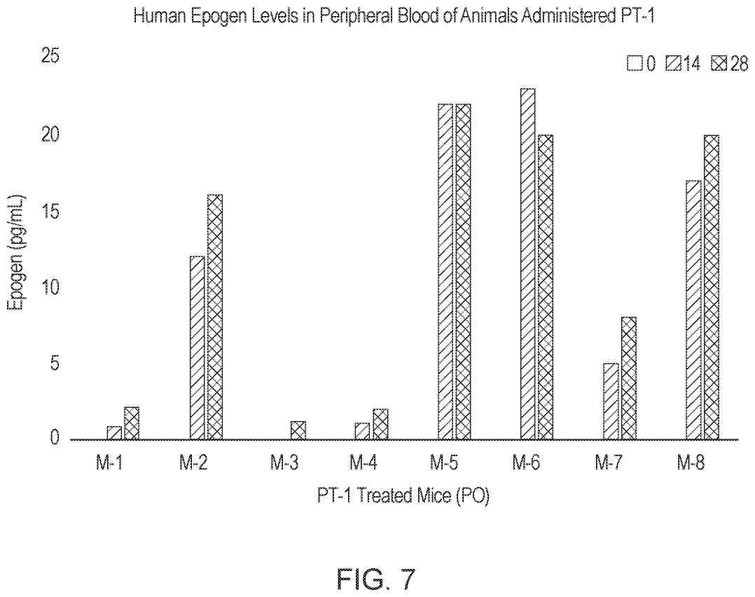

FIG. 7 shows that human erythropoietin is detected in the Sprague Dawley rats (n=8) administered (daily by PO) a composition comprising a polypeptide construct with a sequence identity corresponding to SEQ ID 41, illustrating that the composition is able to traverse the intestinal barrier and deliver erythropoietin to the blood stream of the animals.

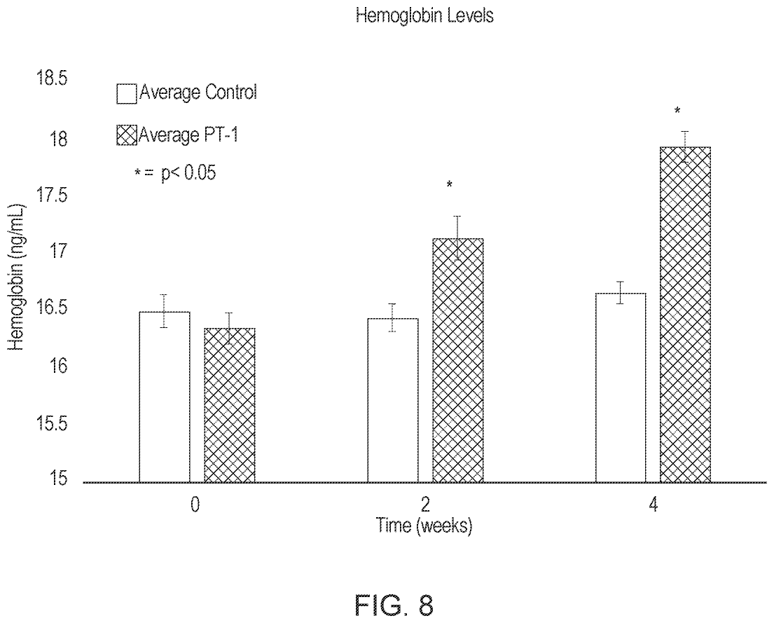

FIG. 8 shows the therapeutic efficacy of a composition comprising a polypeptide construct according to SEQ ID 41 following oral administration to Sprague Dawley rats (600 μg daily by PO). The average hemoglobin levels in the treated animals increased over time (measured in weeks) following oral administration.

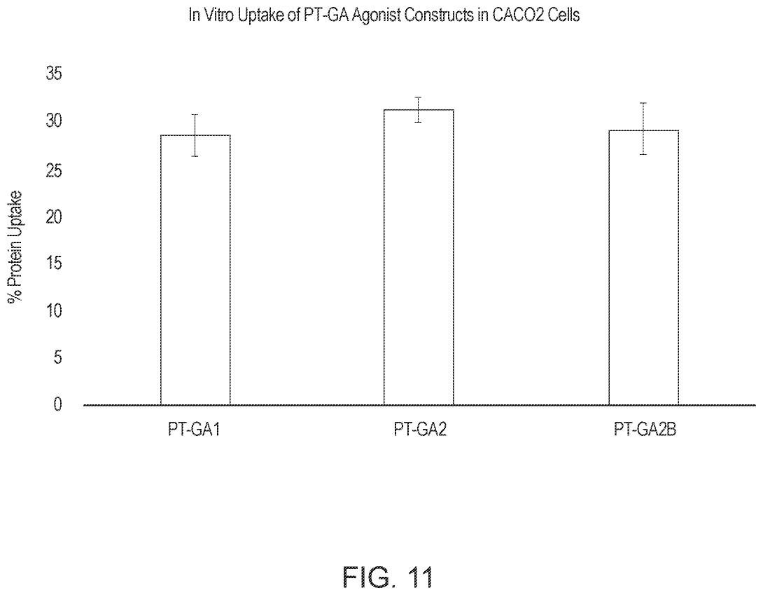

FIG. 11 shows the uptake in a Caco-2 cell model, of a polypeptide construct comprising a polypeptide having a sequence identity according to SEQ ID 1, linked to one or more heterologous polypeptides having a sequence identity according to SEQ ID Nos: 55-56, the heterologous polypeptides comprising GLP-1 agonists (See Example 6). In the figure, exemplary polypeptide constructs include: a polypeptide construct (referred to as “PT-GA1” and corresponding to a polypeptide construct with a sequence identity to SEQ ID NO: 42) comprising a polypeptide according to SEQ ID 1 linked to a heterologous polypeptide comprising a polypeptide according to SEQ ID NO: 55 (an exenatide analog); a polypeptide construct (referred to as “PT-GA2” and corresponding to a polypeptide construct with a sequence identity to SEQ ID NO: 44) comprising a polypeptide according to SEQ ID 1 linked to a heterologous polypeptide comprising a polypeptide according to SEQ ID NO: 56 (a semaglutide/liraglutide analog); and a polypeptide construct comprising a polypeptide according to SEQ ID 1 linked to a semaglutide/liraglutide analog is referenced as PT-GA2 (and corresponds to a polypeptide construct according to SEQ ID 46).

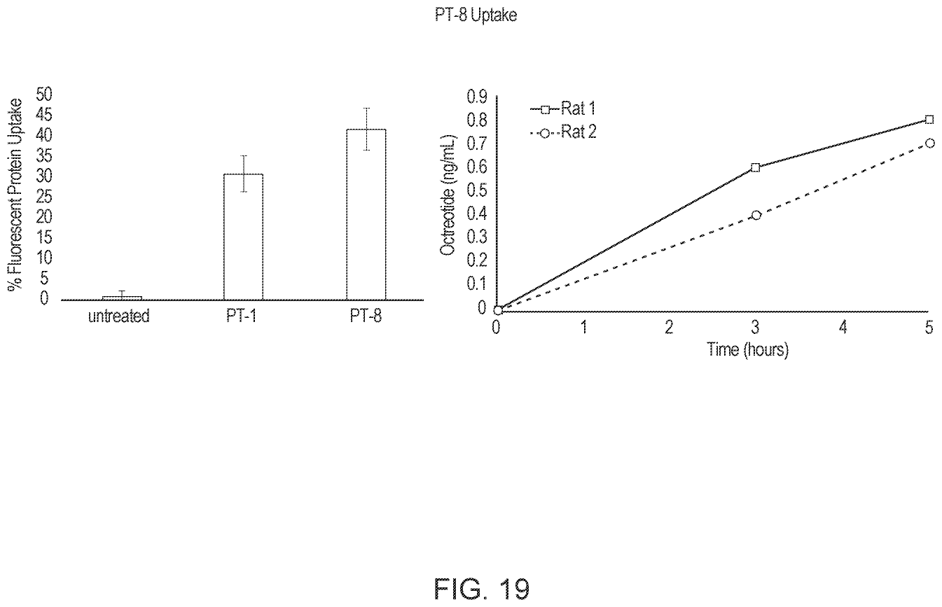

FIG. 19 shows that polypeptide constructs comprising a polypeptide with a sequence identity corresponding to SEQ ID 50 (referenced as PT-OCT) are taken up by Caco-2 cells in vitro and compositions comprising polypeptide constructs comprising a polypeptide with a sequence identity corresponding to SEQ ID 50 provide targeted delivery of a therapeutic polypeptide when administered (PO) in rats (untreated cells and cells treated with a polypeptide construct with a sequence identity corresponding to SEQ ID 41 were used as controls). For in vivo studies, Wistar rats (n=2) were administered (PO) a composition comprising a polypeptide construct with a sequence identity corresponding to SEQ ID 50 at a concentration of 600 μg/animal (in PBS). Blood samples were taken from the animals at 0, 3 and 5 hours post administration. The samples were analyzed and showed increasing levels of a polypeptide fragment according to SEQ ID 53, therefore confirming that orally administered compositions comprising a peptide construct resulted in targeted delivery of a heterologous polypeptide across the gastrointestinal barrier.

### DETAILED DESCRIPTION OF THE DISCLOSURE

Delivering certain therapeutics, including proteins and peptides and other large molecules, by the oral route is extremely challenging for myriad reasons as a result injectable or parenteral administration essentially remains the sole route of administration for certain classes of therapeutics. The very nature of the digestive system is designed to breakdown molecules prior to absorption. The low bioavailability of biologics and peptide-based drugs remains to be an active area of research; the present disclosure provides a promising tool for site-specific drug delivery and improves the oral bioavailability of biologics from less than 1%, to 50%, or more and allows delivery specifically for therapeutics previously not suitable or formulated for oral administration.

The present disclosure provides one or more of the following main advantages to achieve targeted delivery of polypeptide-based therapeutics by the oral route: a) prevents proteolytic activity that degrades the therapeutic in the stomach and gut, b) provides protease-resistant therapeutic polypeptide analogs that retain biological activity, c) stabilize the therapeutic or polypeptide by conjugation to a polypeptide that acts as a “shielding molecule”, and/or d) improve passive therapeutic or polypeptide transport (diffusion) through the epithelial membrane of the intestine.

The present disclosure provides compositions and methods for formulating polypeptide-based therapeutics for oral delivery. Polypeptide based therapeutics have several advantages over small-molecule drugs but are difficult to administer by oral route. First, proteins often serve a highly specific and complex set of functions that cannot be mimicked by simple chemical compounds. Second, because the action of proteins is highly specific, there is often less potential for protein therapeutics to interfere with normal biological processes and cause adverse effects. Third, because the body naturally produces many of the proteins that are used as therapeutics, these agents are often well tolerated and are less likely to elicit immune responses. Fourth, for diseases in which a gene is mutated or deleted, protein therapeutics can provide effective replacement treatment without the need for gene therapy, which is not currently available for most genetic disorders. Fifth, the clinical development and FDA approval time of protein therapeutics may be faster than that of small-molecule drugs.

A relatively small number of protein therapeutics are purified from their native source, such as pancreatic enzymes from hog and pig pancreas and α-1-proteinase inhibitor from pooled human plasma, but most are now produced by recombinant DNA technology and purified from a wide range of organisms. Production systems for recombinant proteins include bacteria, yeast, insect cells, mammalian cells, and transgenic animals and plants. The system of choice can be dictated by the cost of production or the modifications of the protein (for example, glycosylation, phosphorylation or proteolytic cleavage) that are required for biological activity. For example, bacteria do not perform glycosylation reactions, and each of the other biological systems listed above produces a different type or pattern of glycosylation. Protein glycosylation patterns can have a dramatic effect on the activity, half-life and immunogenicity of the recombinant protein in the body. For example, the half-life of native erythropoietin, a growth factor important in erythrocyte production (see below), can be lengthened by increasing the glycosylation of the protein. Darbepoetin-a is an erythropoietin analogue that is engineered to contain two additional amino acids that are substrates for N-linked glycosylation reactions. When expressed in Chinese hamster ovary cells, the analogue is synthesized with five rather than three N-linked carbohydrate chains; this modification causes the half-life of darbepoetin to be threefold longer than that of erythropoietin.

Recombinantly produced proteins can have several further benefits compared with non-recombinant proteins. First, transcription and translation of an exact human gene can lead to a higher specific activity of the protein and a decreased chance of immunological rejection. Second, recombinant proteins are often produced more efficiently and inexpensively, and in potentially limitless quantity. One striking example is found in the protein-based therapy for Gaucher's disease, a chronic congenital disorder of lipid metabolism caused by a deficiency of the enzyme β-glucocerebrosidase (also known as glucosylceramidase) that is characterized by an enlarged liver and spleen, increased skin pigmentation and painful bone lesions. At first, β-glucocerebrosidase purified from human placenta was used to treat this disease, but this requires purification of protein from 50,000 placentas per patient per year, which obviously places a practical limit on the amount of purified protein available. A recombinant form of β-glucocerebrosidase was subsequently developed and introduced, which is not only available in sufficient quantities to treat many more patients with the disease, but also eliminates the risk of transmissible (for example, viral or prion) diseases associated with purifying the protein from human placentas. This also illustrates a third benefit of recombinant proteins over non-recombinant proteins—the reduction of exposure to animal or human diseases.

A fourth advantage is that recombinant technology allows the modification of a protein or the selection of a particular gene variant to improve function or specificity. Again, recombinant β-glucocerebrosidase provides an interesting example. When this protein is made recombinantly, a change of amino-acid arginine to histidine allows the addition of mannose residues to the protein. The mannose is recognized by endocytic carbohydrate receptors on macrophages and many other cell types, allowing the enzyme to enter these cells more efficiently and to cleave the intracellular lipid that has accumulated in pathological amounts, which results in an improved therapeutic outcome. Last, recombinant technology allows the production of proteins that provide a novel function or activity, as discussed below.

Delivering certain therapeutic proteins, including heterologous polypeptides, proteins and peptides and other large molecule therapeutics has been limited to injectable or parenteral administration. Accordingly, provided herein are compositions and methods for targeted delivery of therapeutics across the gastrointestinal lining. In embodiments, the disclosed compositions and methods improve the oral bioavailability of polypeptides and proteins from less than 1% to 50% or more, even for therapeutics previously not considered suitable or formulated for oral administration.

### Definitions

The following terms are used in this disclosure to describe different embodiments. These terms are used for explanation purposes only and are not intended to limit the scope for any aspect of the subject matter claimed herein.

As used herein, “active agent” refers to a biological, chemical or molecular component capable of activity that provides a therapeutic effect.

As used herein “composition” or “formulation” refer (interchangeably) to an active agent in a specific presentation, such as an aqueous solution, solid, semi solid or aerosol for administration by oral or parenteral route. If needed, the formulation may contain pharmaceutically acceptable carriers, excipients and/or one or more additives. The formulations disclosed herein may contain other known active agents, in combination with the active agents described herein.

As used herein, the term “fusion protein” refers to a synthetic, semi-synthetic, or recombinant, protein molecule that comprises all or a portion of two or more different proteins, and/or peptides, and/or polypeptides. For example, provided herein is a fusion protein that comprises a polypeptide and a heterologous polypeptide that are linked to each other. In some embodiments, the fusion protein is synthesized in vitro. In some embodiments, the two or more different polypeptides and/or peptides that the fusion protein is comprised of are produced separately and are subsequently covalently linked. In some embodiments, the fusion protein is expressed as a recombinant protein.

As used herein, an amino acid or nucleotide sequence is “heterologous” to another sequence with which it is operably linked if the two sequences are not associated in nature. Such linkage is not necessarily a covalent linkage. For example, provided herein is a fusion or recombinant protein that comprises a polypeptide and a heterologous polypeptide or protein, wherein the polypeptide and the heterologous polypeptide/protein are not associated in nature. For example, also provided herein is a polypeptide construct that comprises a polypeptide and a heterologous polypeptide.

As used herein the term “linker” refers to a cleavable or non-cleavable linkage between the polypeptide and the heterologous polypeptide. A linker may take many forms, as would be recognized by one of ordinary skill in the art; a linker may be a bond between the two polypeptides, specifically resulting from a bond between atoms of two amino acids or may be a bond formed between atoms of a modification or functional group to one or more amino acids.

As used herein “peptide transporter” or “PT” is nomenclature used to refer to a polypeptide with a sequence identity according to SEQ ID NOs: 1-40.

As used herein “polypeptide” is a polymer of amino acids of three or more amino acids in a serial array, linked through peptide bonds. The term “polypeptide” includes proteins, protein fragments, protein analogues, oligopeptides and the like. The term “polypeptide” contemplates polypeptides that are encoded by nucleic acids, produced through recombinant technology, isolated from an appropriate source, or are synthesized. The term “polypeptide” further contemplates polypeptides as defined above that include chemically modified amino acids or amino acids covalently or noncovalently linked to other molecules, functional groups, ligation ligands, or labeling ligands.

As used herein, the term “polypeptide construct” refers to a synthetic, semi-synthetic, or recombinant single molecule that comprises all or a portion of two or more different proteins and/or polypeptides. For example, provided herein is a polypeptide construct that comprises a first polypeptide and a second polypeptide, wherein the second polypeptide is heterologous to the first polypeptide. The second polypeptide that is heterologous to the first polypeptide is also referred to herein as the “heterologous polypeptide.” In some embodiments, the polypeptide construct is synthesized in vitro. In some embodiments, the two or more different proteins and/or polypeptides that the polypeptide construct is comprised of are produced separately and are subsequently linked.

As used herein “SEQ ID”, or “SEQ ID NO” refer (interchangeably) to a protein, polypeptide, peptide fragment, or analogue thereof, and including any modification thereto, having an amino acid sequence having at least 80%, 85%, 90%, 95%, 98% or 99% sequence identity to the amino acid sequence specified by number, according to the number listed in Table 1.

As used herein, the term “sequence identity” refers to the identity between two nucleic acid molecules, polypeptides, or amino acids, expressed in terms of the identity or similarity between the sequences. Sequence identity can be measured in terms of percentage identity; the higher the percentage, the more identical the sequences are. The percentage identity is calculated over the entire length of the sequence. Homologs or orthologs of amino acid sequences possess a relatively high degree of sequence identity when aligned using standard methods. This homology is more significant when the orthologous proteins are derived from species which are more closely related (e.g., human and mouse sequences), compared to species more distantly related (e.g., human and C. elegans sequences). Methods of alignment of sequences for comparison are well known in the art. Various programs and alignment algorithms are described in: Smith & Waterman; Needleman & Wunsch, J. Mol. Biol. 48:443, 1970; Pearson & Lipman, Proc. Nat. Acad Sci. USA 85:2444, 1988; Higgins & Sharp, Gene, 73:23744, 1988; Higgins & Sharp, CABIOS 5:151-3, 1989; Carpet et al., Nuc. Acids Res. 16:10881-90, 1988; Huang et al. Computer Appls. in the Biosciences 8, 155-65, 1992; and Pearson et al., Meth Mol. Bio. 24:307-31, 1994. Altschul et al., J. Mol. Biol. 215:403-10, 1990, presents a detailed consideration of sequence alignment methods and homology calculations. The level of sequence identity may be determined using The GCG program package (Devereux et al., Nucleic Acids Research 12:387, 1984), BLASTP, BLASTN, FASTA (Altschul et al., J. Mol. Biol. 215:403 (1990), and the ALIGN program (version 2.0). The well-known Smith Waterman algorithm may also be used to determine similarity. The BLAST program is publicly available from NCBI and other sources (BLAST Manual, Altschul, et al., NCBI NLM NIH, Bethesda, Md. 20894; BLAST 2.0 at http://www.ncbi.nlm.nih.gov/blast/). Amino acid residues may be post-translationally modified or conjugated or modified with other functional or non-functional molecular groups; naturally, such modified amino acid residues are included in the amino acid sequences and within the scope of the compositions described herein. For example, polypeptides having at least 80%, at least 85%, at least 90%, at least 95%, at least 98%, or at least 99% identity to specific polypeptides described herein and preferably exhibiting substantially the same functions, as well as polynucleotides encoding such polypeptides, are contemplated. In comparing sequences, the above methods account for various substitutions, deletions, and other modifications. In some embodiments the polypeptide comprises a sequence that has 1, 2, 3, 4, 5, 6, 7, 8, 9, 10 conservative amino acid substitutions as compared any one of SEQ ID NOs: 1-50. As used herein, the terms “conservative amino acid substitutions” and “conservative modifications” refer to amino acid modifications that do not significantly affect or alter the function and/or activity of the presently disclosed proteins comprising the amino acid sequence. Such conservative modifications include amino acid substitutions, additions, and deletions. Modifications can be introduced into the proteins of this disclosure by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively charged amino acids include lysine, arginine, histidine, negatively charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine.

As used herein, “subject” or “individual” or “animal” or “patient” or “mammal” refers to a subject, in particular a mammalian subject, for which treatment is sought, or a diagnosis, prognosis or therapy is desired, for example, to a human.

As used herein a “therapeutic polypeptide” refers to a series of well-ordered amino acids, a protein and/or a polypeptide-based pharmaceutical agent that can be administered to a subject to elicit a biological or medical response of a tissue, system, animal or human that is being sought, for instance, by a researcher or clinician. A therapeutic polypeptide may elicit more than one biological or medical response. A therapeutic polypeptide may be used for therapeutic purposes, i.e., for the treatment of a disorder in a subject. It should be noted that while therapeutic polypeptide may be used for treatment purposes, the disclosure is not limited to such use, as said polypeptide may also be used for in vitro studies. An illustrative, but not exhaustive, example of therapeutic polypeptides is shown in Table 2, which is not intended to limit the scope of the disclosure or interpretation of the claims.

As used herein, the terms “treat,” “treating” or “treatment,” and other grammatical equivalents as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and prophylaxis. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disorder, or to a patient reporting one or more of the physiological symptoms, even though a diagnosis may not have been made.

As used herein, a “therapeutically effective amount” or “effective amount”, is an amount of biologically active agent/therapeutic polypeptide capable of achieving a clinically relevant endpoint in a subject when administered in one or repeated doses to the subject. Such effect need not be absolute to be beneficial. The appropriate dose of the composition may depend on the route of administration, such as oral, injection or infusion, and may depend on the subject being treated as well as the severity of the condition to be treated. Using scaling methods, such as allometric scaling, it is possible to predict suitable and exemplary dosage ranges for the administration of compositions, as disclosed herein, to adult humans. Dose scaling is an empirical approach, is well characterized and understood in the art. This approach assumes that there are some unique characteristics on anatomical, physiological, and biochemical process among species, and the possible difference in pharmacokinetics/physiological time is, as such, accounted for by scaling. Determination of a therapeutically effective amount is well within the capability of those skilled in the art, especially in light of the detailed disclosure provided herein.

As used herein, “vector” is a nucleic acid molecule, preferably self-replicating in an appropriate host, which transfers an inserted nucleic acid molecule into and/or between host cells. The term includes vectors that function primarily for insertion of DNA or RNA into a cell, replication of vectors that function primarily for the replication of DNA or RNA, and expression vectors that function for transcription and/or translation of the DNA or RNA. Also included are vectors that provide more than one of the above functions. An “expression vector” is a polynucleotide which, when introduced into an appropriate host cell, can be transcribed, and translated into a polypeptide(s). An “expression system” usually connotes a suitable host cell comprised of an expression vector that can function to yield a desired expression product.

Disclosed herein are polypeptides, polypeptide fragments, heterologous polypeptides, and polypeptide constructs formed therefrom, with sequence identities corresponding to SEQ ID NOs: 1-59, as identified and set forth in Table 1.

TABLE 1

| SEQ ID | | |
|---|---|---|
| NO: | Amino Acid Sequence | Description |
| 1 | MADDAGAAGGPGGPGGPGMGNRGGFRGGFG | Exemplary polypeptide sequence (also |
| | SGIRGRGRGRGRGRGRGRGA | referred to herein as peptide |
| | | transporter, or “PT”) |
| 2 | ADDAGAAGGPGGPGGPGMGNRGGFRGGFGS | Exemplary polypeptide sequence (also |
| | GIRGRGRGRGRGRGRGRGA | referred to herein as peptide |
| | | transporter, or “PT”) |
| 3 | DDAGAAGGPGGPGGPGMGNRGGFRGGFGSGI | Exemplary polypeptide sequence (also |
| | RGRGRGRGRGRGRGRGA | referred to herein as peptide |
| | | transporter, or “PT”) |
| 4 | DAGAAGGPGGPGGPGMGNRGGFRGGFGSGIR | Exemplary polypeptide sequence (also |
| | GRGRGRGRGRGRGRGA | referred to herein as peptide |
| | | transporter, or “PT”) |
| 5 | AGAAGGPGGPGGPGMGNRGGFRGGFGSGIR | Exemplary polypeptide sequence (also |
| | GRGRGRGRGRGRGRGA | referred to herein as peptide |
| | | transporter, or “PT”) |
| 6 | GAAGGPGGPGGPGMGNRGGFRGGFGSGIRG | Exemplary polypeptide sequence (also |
| | RGRGRGRGRGRGRGA | referred to herein as peptide |
| | | transporter, or “PT”) |
| 7 | AAGGPGGPGGPGMGNRGGFRGGFGSGIRGR | Exemplary polypeptide sequence (also |
| | GRGRGRGRGRGRGA | referred to herein as peptide |
| | | transporter, or “PT”) |
| 8 | AGGPGGPGGPGMGNRGGFRGGFGSGIRGRG | Exemplary polypeptide sequence (also |
| | RGRGRGRGRGRGA | referred to herein as peptide |
| | | transporter, or “PT”) |
| 9 | GGPGGPGGPGMGNRGGFRGGFGSGIRGRGR | Exemplary polypeptide sequence (also |
| | GRGRGRGRGRGA | referred to herein as peptide |
| | | transporter, or “PT”) |
| 10 | GPGGPGGPGMGNRGGFRGGFGSGIRGRGRG | Exemplary polypeptide sequence (also |
| | RGRGRGRGRGA | referred to herein as peptide |
| | | transporter, or “PT”) |
| 11 | PGGPGGPGMGNRGGFRGGFGSGIRGRGRGR | Exemplary polypeptide sequence (also |
| | GRGRGRGRGA | referred to herein as peptide |
| | | transporter, or “PT”) |
| 12 | GGPGGPGMGNRGGFRGGFGSGIRGRGRGRG | Exemplary polypeptide sequence (also |
| | RGRGRGRGA | referred to herein as peptide |
| | | transporter, or “PT”) |
| 13 | GPGGPGMGNRGGFRGGFGSGIRGRGRGRGR | Exemplary polypeptide sequence (also |
| | GRGRGRGA | referred to herein as peptide |
| | | transporter, or “PT”) |
| 14 | PGGPGMGNRGGFRGGFGSGIRGRGRGRGRG | Exemplary polypeptide sequence (also |
| | RGRGRGA | referred to herein as peptide |
| | | transporter, or “PT”) |
| 15 | GGPGMGNRGGFRGGFGSGIRGRGRGRGRGR | Exemplary polypeptide sequence (also |
| | GRGRGA | referred to herein as peptide |
| | | transporter, or “PT”) |
| 16 | GPGMGNRGGFRGGFGSGIRGRGRGRGRGRG | Exemplary polypeptide sequence (also |
| | RGRGA | referred to herein as peptide |
| | | transporter, or “PT”) |
| 17 | PGMGNRGGFRGGFGSGIRGRGRGRGRGRGR | Exemplary polypeptide sequence (also |
| | GRGA | referred to herein as peptide |
| | | transporter, or “PT”) |
| 18 | GMGNRGGFRGGFGSGIRGRGRGRGRGRGRG | Exemplary polypeptide sequence (also |
| | RGA | referred to herein as peptide |
| | | transporter, or “PT”) |
| 19 | MGNRGGFRGGFGSGIRGRGRGRGRGRGRGR | Exemplary polypeptide sequence (also |
| | GA | referred to herein as peptide |
| | | transporter, or “PT”) |
| 20 | GNRGGFRGGFGSGIRGRGRGRGRGRGRGRG | Exemplary polypeptide sequence (also |
| | A | referred to herein as peptide |
| | | transporter, or “PT”) |
| 21 | MADDAGAAGGPGGPGGPGMGNRGGFRGGFG | Exemplary polypeptide sequence (also |
| | SGIRGRGRGRGRGRGRGRGK | referred to herein as peptide |
| | | transporter, or “PT”) with modified |
| | | Lys(N3) for ligation (bold)) |
| 22 | ADDAGAAGGPGGPGGPGMGNRGGFRGGFGS | Exemplary polypeptide sequence (also |
| | GIRGRGRGRGRGRGRGRGK | referred to herein as peptide |
| | | transporter, or “PT”) with modified |
| | | Lys(N3) for ligation (bold)) |
| 23 | DDAGAAGGPGGPGGPGMGNRGGFRGGFGSGI | Exemplary polypeptide sequence (also |
| | RGRGRGRGRGRGRGRGK | referred to herein as peptide |
| | | transporter, or “PT”) with modified |
| | | Lys(N3) for ligation (bold) |
| 24 | DAGAAGGPGGPGGPGMGNRGGFRGGFGSGIR | Exemplary polypeptide sequence (also |
| | GRGRGRGRGRGRGRGK | referred to herein as peptide |
| | | transporter, or “PT”) with modified |
| | | Lys(N3) for ligation (bold)) |
| 25 | AGAAGGPGGPGGPGMGNRGGFRGGFGSGIR | Exemplary polypeptide sequence (also |
| | GRGRGRGRGRGRGRGK | referred to herein as peptide |
| | | transporter, or “PT”) with modified |
| | | Lys(N3) for ligation (bold)) |
| 26 | GAAGGPGGPGGPGMGNRGGFRGGFGSGIRG | Exemplary polypeptide sequence (also |
| | RGRGRGRGRGRGRGK | referred to herein as peptide |
| | | transporter, or “PT”) with modified |
| | | Lys(N3) for ligation (bold)) |
| 27 | AAGGPGGPGGPGMGNRGGFRGGFGSGIRGR | Exemplary polypeptide sequence (also |
| | GRGRGRGRGRGRGK | referred to herein as peptide |
| | | transporter, or “PT”) with modified |
| | | Lys(N3) for ligation (bold)) |
| 28 | AGGPGGPGGPGMGNRGGFRGGFGSGIRGRG | Exemplary polypeptide sequence (also |
| | RGRGRGRGRGRGK | referred to herein as peptide |
| | | transporter, or “PT”) with modified |
| | | Lys(N3) for ligation (bold)) |
| 29 | GGPGGPGGPGMGNRGGFRGGFGSGIRGRGR | Exemplary polypeptide sequence (also |
| | GRGRGRGRGRGK | referred to herein as peptide |
| | | transporter, or “PT”) with modified |
| | | Lys(N3) for ligation (bold)) |
| 30 | GPGGPGGPGMGNRGGFRGGFGSGIRGRGRG | Exemplary polypeptide sequence (also |
| | RGRGRGRGRGK | referred to herein as peptide |
| | | transporter, or “PT”) with modified |
| | | Lys(N3) for ligation (bold)) |
| 31 | PGGPGGPGMGNRGGFRGGFGSGIRGRGRGR | Exemplary polypeptide sequence (also |
| | GRGRGRGRGK | referred to herein as peptide |
| | | transporter, or “PT”) with modified |
| | | Lys(N3) for ligation (bold)) |
| 32 | GGPGGPGMGNRGGFRGGFGSGIRGRGRGRG | Exemplary polypeptide sequence (also |
| | RGRGRGRGK | referred to herein as peptide |
| | | transporter, or “PT”) with modified |
| | | Lys(N3) for ligation (bold)) |
| 33 | GPGGPGMGNRGGFRGGFGSGIRGRGRGRGR | Exemplary polypeptide sequence (also |
| | GRGRGRGK | referred to herein as peptide |
| | | transporter, or “PT”) with modified |
| | | Lys(N3) for ligation (bold)) |
| 34 | PGGPGMGNRGGFRGGFGSGIRGRGRGRGRG | Exemplary polypeptide sequence (also |
| | RGRGRGK | referred to herein as peptide |
| | | transporter, or “PT”) with modified |
| | | Lys(N3) for ligation (bold)) |
| 35 | GGPGMGNRGGFRGGFGSGIRGRGRGRGRGR | Exemplary polypeptide sequence (also |
| | GRGRGK | referred to herein as peptide |
| | | transporter, or “PT”) with modified |
| | | Lys(N3) for ligation (bold)) |
| 36 | GPGMGNRGGFRGGFGSGIRGRGRGRGRGRG | Exemplary polypeptide sequence (also |
| | RGRGK | referred to herein as peptide |
| | | transporter, or “PT”) with modified |
| | | Lys(N3) for ligation (bold)) |
| 37 | PGMGNRGGFRGGFGSGIRGRGRGRGRGRGR | Exemplary polypeptide sequence (also |
| | GRGK | referred to herein as peptide |
| | | transporter, or “PT”) with modified |
| | | Lys(N3) for ligation (bold)) |
| 38 | GMGNRGGFRGGFGSGIRGRGRGRGRGRGRG | Exemplary polypeptide sequence (also |
| | RGK | referred to herein as peptide |
| | | transporter, or “PT”) with modified |
| | | Lys(N3) for ligation (bold)) |
| 39 | MGNRGGFRGGFGSGIRGRGRGRGRGRGRGR | Exemplary polypeptide sequence (also |
| | GK | referred to herein as peptide |
| | | transporter, or “PT”) with modified |
| | | Lys(N3) for ligation (bold)) |
| 40 | GNRGGFRGGFGSGIRGRGRGRGRGRGRGRG | Exemplary polypeptide sequence (also |
| | K | referred to herein as peptide |
| | | transporter, or “PT”) with modified |
| | | Lys(N3) for ligation (bold)) |
| 41 | MADDAGAAGGPGGPGGPGMGNRGGFRGGFG | Polypeptide construct, designated PT- |
| | SGIRGRGRGRGRGRGRGRGAAPPRLICDSRVL | EPO, |
| | ERYLLEAKEAENITTGCAEHCSLNENITVPDTKV | comprising SEQ ID NO: 1 (bold) and |
| | NFYAWKRMEVGQQAVEVWQGLALLSEAVLRG | human erythropoietin according to SEQ |
| | QALLVNSSQPWEPLQLHVDKAVSGLRSLTTLLR | ID 51 |
| | ALGAQKEAISPPDAASAAPLRTITADTFRKLFRV | |
| | YSNFLRGKLKLYTGEACRTGDR | |
| 42 | MADDAGAAGGPGGPGGPGMGNRGGFRGGFG | Polypeptide construct designated PT- |
| | SGIRGRGRGRGRGRGRGRGAHGEGTFTSDLS | GA-1 |
| | KQMEEEAVRLFIEWLKNGGPSSGAPPPS | comprising SEQ ID NO: 1 (bold) linked |
| | | to exenatide (synthetic exendin-4) |
| 43 | MADDAGAAGGPGGPGGPGMGNRGGFRGGFG | Polypeptide construct designated PT- |
| | SGIRGRGRGRGRGRGRGRGXaaHGEGTFTSDL | GA-1 |
| | SKQMEEEAVRLFIEWLKNGGPSSGAPPPS | comprising SEQ ID NO: 21 (bold) ligated |
| | | to exenatide (synthetic exendin-4); |
| | | Where Xaa is Nle (amide bond formed |
| | | from click ligation) |
| 44 | MADDAGAAGGPGGPGGPGMGNRGGFRGGFG | Polypeptide construct designated PT- |
| | SGIRGRGRGRGRGRGRGRGAHAEGTFTSDVS | GA2 |
| | SYLEGQAAKEFIAWLVRGRG | comprising SEQ ID NO: 1 (bold) linked |
| | | to liraglutide analog (a GLP-1 agonist) |
| 45 | MADDAGAAGGPGGPGGPGMGNRGGFRGGFG | Polypeptide construct designated PT- |
| | SGIRGRGRGRGRGRGRGRGXaaHAEGTFTSDV | GA2 |
| | SSYLEGQAAKEFIAWLVRGRG | comprising SEQ ID NO: 21 (bold) ligated |
| | | to liraglutide analog (a GLP-1 agonist) |
| | | Where Xaa is Nle (amide bond formed |
| | | from click ligation) |
| 46 | MADDAGAAGGPGGPGGPGMGNRGGFRGGFG | Polypeptide construct designated PT- |
| | SGIRGRGRGRGRGRGRGRGAHAEGTFTSDVS | GA2B |
| | SYLEGQAAKEFIAWLVRGRGMADDAGAAGGP | comprising SEQ ID NO: 1 (bold), |
| | GGPGGPGMGNRGGFRGGFGSGIRGRGRGRG | liraglutide (an GLP-1 analog), and SEQ |
| | RGRGRGRGA | ID NO: 1 (bold) at both C-terminus and |
| | | N-terminus of the heterologous |
| | | polypeptide |
| 47 | MADDAGAAGGPGGPGGPGMGNRGGFRGGFG | Polypeptide construct comprising SEQ |
| | SGIRGRGRGRGRGRGRGRGAHDEFERHAEGT | ID NO: 1 linked to GLP-1 (human) |
| | FTSDVSSYLEGQAAKEFIAWLVKGR | |
| 48 | MADDAGAAGGPGGPGGPGMGNRGGFRGGFG | Polypeptide construct comprising SEQ |
| | SGIRGRGRGRGRGRGRGRGXaaHDEFERHAE | ID NO: 1 ligated to GLP-1 (human); |
| | GTFTSDVSSYLEGQAAKEFIAWLVKGR | Where Xaa is Nle (amide bond formed |
| | | from click ligation) |
| 49 | MADDAGAAGGPGGPGGPGMGNRGGFRGGFG | Polypeptide construct designated PT- |
| | SGIRGRGRGRGRGRGRGRGAFCFWKTCT | OCT comprising SEQ ID NO: 1 (bold) |
| | | and octapeptide (octreotide) according |
| | | to SEQ ID 52 |
| 50 | MADDAGAAGGPGGPGGPGMGNRGGFRGGFG | Polypeptide construct designated PT- |
| | SGIRGRGRGRGRGRGRGRGXaaFCFWKTCT | OCT comprising SEQ ID NO: 21 (bold) |
| | | ligated to an octapeptide (Pentynoyl- |
| | | Octreotide)-an octreotide/somatostatin |
| | | analogue, corresponding to a modified |
| | | peptide according to SEQ ID 53; Where |
| | | Xaa is Nle (amide bond formed from |
| | | click ligation) |
| 51 | APPRLICDSRVLERYLLEAKEAENITTGCAEHCS | Heterologous polypeptide comprising |
| | LNENITVPDTKVNFYAWKRMEVGQQAVEVWQG | Erythropoietin sequence |
| | LALLSEAVLRGQALLVNSSQPWEPLQLHVDKAV | |
| | SGLRSLTTLLRALGAQKEAISPPDAASAAPLRTIT | |
| | ADTFRKLFRVYSNFLRGKLKLYTGEACRTGDR | |
| 52 | FCFWKTCT | Heterologous polypeptide comprising |
| | | Octreotide sequence |
| 53 | HGEGTFTSDLSKQMEEEAVRLFIEWLKNGGPSS | Heterologous polypeptide comprising |
| | GAPPPS | Exenatide sequence |
| 54 | HAEGTFTSDVSSYLEGQAAK*EEFIAWLVRGRG | Heterologous polypeptide comprising |
| | (where K* is substituted at Nϵ- | Liraglutide/Semaglutide sequence |
| | position with (γ-glutamyl | |
| | (Nα-hexadecanoyl)). | |
| 55 | HDEFERHAEGTFTSDVSSYLEGQAAKEFIAWLV | Heterologous polypeptide comprising |
| | KGR | GLP-1 sequence |
| 56 | GAAPPRLICDSRVLERYLLEAKEAENITTGCAEH | Erythropoietin sequence with N-terminal |
| | CSLNENITVPDTKVNFYAWKRMEVGQQAVEVW | “GA” following cleavage of PT (post oral |
| | QGLALLSEAVLRGQALLVNSSQPWEPLQLHVDK | administration) |
| | AVSGLRSLTTLLRALGAQKEAISPPDAASAAPLR | |
| | TITADTFRKLFRVYSNFLRGKLKLYTGEACRTGD | |
| | R | |
| 57 | MADDAGAAGGPGGPGGPGMGNRGGFRGGFG | Polypeptide fragment of SEQ ID NO: 1 |
| | SGIRGRGRGRGRGRGRGR | (post oral administration) |
| 58 | Xaa01Xaa02Xaa03Xaa04Xaa05Xaa06Xaa07 | Polypeptide motif sequence |
| | Xaa08Xaa09Xaa10Xaa11Xaa12Xaa13Xaa14 | Xaa01 = M, A, V, I, L |
| | Xaa15Xaa16Xaa17Xaa18Xaa19Xaa20Xaa21 | Xaa02 = A, G, S |
| | Xaa22Xaa23Xaa24Xaa25Xaa26Xaa27Xaa28 | Xaa03 = D, E |
| | Xaa29Xaa30Xaa31Xaa32Xaa33Xaa34Xaa35 | Xaa04 = D, E |
| | Xaa36Xaa37Xaa38Xaa39Xaa40Xaa41Xaa42 | Xaa05 = A, G, S |
| | Xaa43Xaa44Xaa45Xaa46Xaa47Xaa48Xaa49 | Xaa06 = G, A, S |
| | Xaa50 | Xaa07 = A, G, S |
| | | Xaa08 = A, G, S |
| | | Xaa09 = G, A, S |
| | | Xaa10 = G, A, S |
| | | Xaa11 = P |
| | | Xaa12 = G, A, S |
| | | Xaa13 = G, A, S |
| | | Xaa14 = P |
| | | Xaa15 = G, A, S |
| | | Xaa16 = G, A, S |
| | | Xaa17 = P |
| | | Xaa18 = G, A, S |
| | | Xaa19 = M, A, V, I, L |
| | | Xaa20 = M, T, I, G, A, S |
| | | Xaa21 = N, G, A, Q |
| | | Xaa22 = N, Q, R, K |
| | | Xaa23 = R, K, G, A, S |
| | | Xaa24 = G, A, S |
| | | Xaa25 = G, A, F, Y, W, H |
| | | Xaa26 = F, Y, W, R, K |
| | | Xaa27 = R, K, G, A, S |
| | | Xaa28 = G, A, S |
| | | Xaa29 = G, A, F, Y, W, H |
| | | Xaa30 = F, Y, W, G, A, S |
| | | Xaa31 = G, A, S |
| | | Xaa32 = S, T, G, A |
| | | Xaa33 = G, A, I, V, L, M |
| | | Xaa34 = R, K |
| | | Xaa35 = G, A, S |
| | | Xaa36 = R, K |
| | | Xaa37 = G, A, S |
| | | Xaa38 = R, K |
| | | Xaa39 = G, A, S |
| | | Xaa40 = R, K |
| | | Xaa41 = G, A, S |
| | | Xaa42 = R, K |
| | | Xaa43 = G, A, S |
| | | Xaa44 = R, K |
| | | Xaa45 = G, A, S |
| | | Xaa46 = R, K |
| | | Xaa47 = G, A, S |
| | | Xaa48 = R, K |
| | | Xaa49 = G, A, S |
| | | Xaa50 = A, G, |
| 59 | Xaa01Xaa02Xaa03Xaa04Xaa05Xaa06Xaa07 | Polypeptide motif sequence |
| | Xaa08Xaa09Xaa10Xaa11Xaa12Xaa13Xaa14 | Xaa01 = M, A, V, I, L |
| | Xaa15Xaa16Xaa17Xaa18Xaa19Xaa20Xaa21 | Xaa02 = A, G, S |
| | Xaa22Xaa23Xaa24Xaa25Xaa26Xaa27Xaa28 | Xaa03 = D, E |
| | Xaa29Xaa30Xaa31Xaa32Xaa33Xaa34Xaa35 | Xaa04 = D, E |
| | Xaa36Xaa37Xaa38Xaa39Xaa40Xaa41Xaa42 | Xaa05 = A, G, S |
| | Xaa43Xaa44Xaa45Xaa46Xaa47Xaa48Xaa49 | Xaa06 = G, A, S |
| | Xaa50Xaa51 | Xaa07 = A, G, S |
| | | Xaa08 = A, G, S |
| | | Xaa09 = G, A, S |
| | | Xaa10 = G, A, S |
| | | Xaa11 = P |
| | | Xaa12 = G, A, S |
| | | Xaa13 = G, A, S |
| | | Xaa14 = P |
| | | Xaa15 = G, A, S |
| | | Xaa16 = G, A, S |
| | | Xaa17 = P |
| | | Xaa18 = G, A, S |
| | | Xaa19 = M, A, V, I, L |
| | | Xaa20 = M, T, I, G, A, S |
| | | Xaa21 = N, G, A, Q |
| | | Xaa22 = N, Q, R, K |
| | | Xaa23 = R, K, G, A, S |
| | | Xaa24 = G, A, S |
| | | Xaa25 = G, A, F, Y, W, H |
| | | Xaa26 = F, Y, W, R, K |
| | | Xaa27 = R, K, G, A, S |
| | | Xaa28 = G, A, S |
| | | Xaa29 = G, A, F, Y, W, H |
| | | Xaa30 = F, Y, W, G, A, S |
| | | Xaa31 = G, A, S |
| | | Xaa32 = S, T, G, A |
| | | Xaa33 = G, A, I, V, L, M |
| | | Xaa34 = R, K |
| | | Xaa35 = G, A, S |
| | | Xaa36 = R, K |
| | | Xaa37 = G, A, S |
| | | Xaa38 = R, K |
| | | Xaa39 = G, A, S |
| | | Xaa40 = R, K |
| | | Xaa41 = G, A, S |
| | | Xaa42 = R, K |
| | | Xaa43 = G, A, S |
| | | Xaa44 = R, K |
| | | Xaa45 = G, A, S |
| | | Xaa46 = R, K |
| | | Xaa47 = G, A, S |
| | | Xaa48 = R, K |
| | | Xaa49 = G, A, S |
| | | Xaa50 = A, G, S |
| | | Xaa51 = K |

A composition is disclosed, comprising a polypeptide according to the formula:

Xaa01Xaa02Xaa03Xaa04Xaa05Xaa06Xaa07Xaa08Xaa09Xaa10
Xaa11Xaa12Xaa13Xaa14Xaa15Xaa16Xaa17Xaa18Xaa19Xaa20
Xaa21Xaa22Xaa23Xaa24Xaa25Xaa26Xaa27Xaa28Xaa29Xaa30
Xaa31Xaa32Xaa33Xaa34Xaa35Xaa36Xaa37Xaa38Xaa39Xaa40
Xaa41Xaa42Xaa43Xaa44Xaa45Xaa46Xaa47Xaa48Xaa49Xaa50

### Wherein:

Xaa01=M, A, V, I, L
Xaa02=A, G, S
Xaa03=D, E
Xaa04=D, E
Xaa05=A, G, S
Xaa06=G, A, S
Xaa07=A, G, S
Xaa08=A, G, S
Xaa09=G, A, S
Xaa10=G, A, S
Xaa11=P
Xaa12=G, A, S
Xaa13=G, A, S
Xaa14=P
Xaa15=G, A, S
Xaa16=G, A, S
Xaa17=P
Xaa18=G, A, S
Xaa19=M, A, V, I, L
Xaa20=M, T, I, G, A, S
Xaa21=N, G, A, Q
Xaa22=N, Q, R, K
Xaa23=R, K, G, A, S
Xaa24=G, A, S
Xaa25=G, A, F, Y, W, H
Xaa26=F, Y, W, R, K
Xaa27=R, K, G, A, S
Xaa28=G, A, S
Xaa29=G, A, F, Y, W, H
Xaa30=F, Y, W, G, A, S
Xaa31=G, A, S
Xaa32=S, T, G, A
Xaa33=G, A, I, V, L, M
Xaa34=R, K
Xaa35=G, A, S
Xaa36=R, K
Xaa37=G, A, S
Xaa38=R, K
Xaa39=G, A, S
Xaa40=R, K
Xaa41=G, A, S
Xaa42=R, K
Xaa43=G, A, S
Xaa44=R, K
Xaa45=G, A, S
Xaa46=R, K
Xaa47=G, A, S
Xaa48=R, K
Xaa49=G, A, S
Xaa50=A, G, S, K

A compound is disclosed, comprising a polypeptide of the formula:

H-Met-Ala-Asp-Asp-Ala⁵-Gly-Ala-Ala-Gly-Gly¹⁰-Pro-
Gly-Gly-Pro-Gly¹⁵-Gly-Pro-Gly-Met-Gly²⁰-Asn-Arg-
Gly-Gly-Phe²⁵-Arg-Gly-Gly-Phe-Gly³⁰-Ser-Gly-Ile-
Arg-Gly₃₅-Arg-Gly-Arg-Gly-Arg⁴⁰-Gly-Arg-Gly-Arg-
Gly⁴⁵-Arg-Gly-Arg-Gly-Lys(N3)⁵⁰-OH;

wherein the polypeptide is capable of being linked, via the Lysine terminal residue, to a heterologous polypeptide; and wherein the compound provides targeted delivery of the heterologous polypeptide when administered to a subject.

Disclosed herein is a heterologous polypeptide according to SEQ ID 52 modified for ligation to a polypeptide, comprising: Propynoic Acid-D-Phe-Cys-Phe-D-Trp-Lys-Thr-Cys-Thr-ol, wherein the modified heterologous polypeptide is configured for conjugation to a polypeptide, wherein the polypeptide is modified with a terminus comprising a modified lysine residue comprising Lys(N3)⁵⁰-OH.

Disclosed herein is a compound comprising a formula comprising H-Met-Ala-Asp-Asp-Ala-Gly-Ala-Ala-Gly-Gly-Pro-Gly-Gly-Pro-Gly-Gly-Pro-Gly-Met-Gly-Asn-Arg-Gly-Gly-Phe-Arg-Gly-Gly-Phe-Gly-Ser-Gly-Ile-Arg-Gly-Arg-Gly-Arg-Gly-Arg-Gly-Arg-Gly-Arg-Gly-Arg-Gly-Arg-Gly-Nle (triazol-propionyl-D-Phe-Cys-Phe-D-Tru-Lys-Thr-Cys-Thr-ol)-OH, wherein the compound provides targeted delivery of the polypeptide when administered to a subject by oral route.

Disclosed herein are polypeptide constructs suitable for delivering a heterologous polypeptide across the gastrointestinal lining, when administered by oral route to a subject, the peptide constructs comprising a polypeptide linked to the heterologous polypeptide, wherein the polypeptide is a polypeptide having at least 90% sequence identity to a peptide according to SEQ ID 1-40, wherein the heterologous polypeptide is a therapeutic polypeptide, and wherein the polypeptide is joined to the heterologous polypeptide by a linker.

The polypeptide and heterologous polypeptide may be linked directly or indirectly through a covalent and/or an ionic bond. In one aspect, the polypeptide construct comprises a polypeptide and a heterologous polypeptide. In embodiments, the polypeptide and the heterologous polypeptide are linked by an ionic bond. An ionic bond refers to a linkage that results from the electrostatic attraction between oppositely charged ions. In embodiments, the polypeptide and the heterologous polypeptide are linked by a covalent bond. A covalent bond refers the mutual sharing of one or more pairs of electrons between two atoms. In one aspect, the polypeptide and heterologous polypeptide are linked by an amide bond or peptide bond.

In some embodiments, the polypeptide is linked to the N-terminus of the heterologous polypeptide. In some embodiments, the polypeptide is linked to the C-terminus of the heterologous polypeptide. In some embodiments, the C-terminus of the polypeptide is linked to the N-terminus of the heterologous polypeptide. In some embodiments, the N-terminus of the polypeptide is linked to the C-terminus of the heterologous polypeptide. In some embodiments, the N-terminus of the heterologous polypeptide is linked to the N-terminus of the polypeptide. In some embodiments, the C-terminus of the heterologous polypeptide is linked to the C-terminus of the polypeptide. The term “linked” does not necessarily require that the polypeptide and the heterologous polypeptide are linked directly to each other. In embodiments, the polypeptide and the heterologous polypeptide are linked through a linker such as an additional moiety, which may be cleavable or non-cleavable.

The polypeptide construct may comprise two or more polypeptides and a heterologous polypeptide. In some embodiments, the polypeptide construct comprises at least the following components in the indicated orientation: polypeptide-heterologous polypeptide-polypeptide. In some embodiments, the polypeptide construct comprises at least the following components in the indicated orientation: polypeptide-polypeptide-heterologous polypeptide. In some embodiments, the polypeptide construct comprises at least the following components in the indicated orientation: heterologous polypeptide-polypeptide-polypeptide.

In some embodiments, the polypeptide is linked to the heterologous polypeptide through a linker. In embodiments, the linker is a polypeptide linker at least 1, at least 2, at least 3, at least 5, at least 7, at least 10 amino acid acids long. In embodiments, the polypeptide linker is between 1 and 20 amino acid acids long. The linker may comprise natural and non-naturally occurring amino acids. The linker linking the polypeptide and the heterologous polypeptide may comprise flexible and/or rigid portions. In embodiments, the linker is a flexible linker. In embodiments, the linker is a rigid linker. In embodiments, the linker is a polypeptide linker comprising one or more, or a plurality of, glycines and serines. In one embodiment, the linker is a cleavable linker. In one embodiment the linker is a lysine or plurality of lysine residues. In one embodiment, the linker is a non-cleavable linker. In one embodiment, the linker is a helical linker. In one embodiment, the linker is a non-helical linker. In one embodiment, the linker is a strong non-covalent interaction, such as biotin-streptavidin. In one embodiment, the linker is an amide bond formed between an alkyl modified peptide on the polypeptide and an azide modified peptide on the heterologous polypeptide.

In one embodiment, some or all the components making up the polypeptide construct are produced separately, for example by recombinant expression or by chemical synthesis, and are joined subsequently. In embodiments, some or all the components making up the polypeptide construct, or the entire polypeptide construct are produced in a recombinant host cell or are synthesized from a recombinant nucleic acid. A “recombinant host cell” is a host cell that comprises a recombinant nucleic acid. The term “recombinant nucleic acid” as used herein refers to a nucleic acid that is removed from its naturally occurring environment, or a nucleic acid that is not associated with all or a portion of a nucleic acid abutting or proximal to the nucleic acid when it is found in nature, or a nucleic acid that is operatively linked to a nucleic acid that it is not linked to in nature, or a nucleic acid that does not occur in nature, or a nucleic acid that contains a modification that is not found in that nucleic acid in nature (e.g., insertion, deletion, or point mutation introduced artificially, e.g., by human intervention), or a nucleic acid that is integrated into a chromosome at a heterologous site. The term includes cloned DNA isolates and nucleic acids that comprise chemically synthesized nucleotide analogs.

A variety of expression vectors have been developed for the efficient synthesis polypeptide constructs in prokaryotic cells such as bacteria and in eukaryotic systems, including but not limited to yeast and mammalian cell culture systems have been developed. The vectors can comprise segments of chromosomal, non-chromosomal and synthetic DNA sequences. Also provided are cells comprising expression vectors for the expression of the polypeptide constructs disclosed herein. Expression vectors are typically replicable in the host organisms either as episomes or as an integral part of the host chromosomal DNA. Commonly, expression vectors contain selection markers (e.g., ampicillin-resistance, hygromycin-resistance, tetracycline resistance or neomycin resistance) to permit detection of those cells transformed with the desired DNA sequences (see, e.g., Itakura et al., U.S. Pat. No. 4,704,362).

The expression of the polypeptide constructs disclosed herein can occur in either prokaryotic or eukaryotic cells. Suitable hosts include bacterial or eukaryotic hosts, including yeast, insects, fungi, bird and mammalian cells either in vivo, or in situ, or host cells of mammalian, insect, bird or yeast origin. The mammalian cell or tissue can be of human, primate, hamster, rabbit, rodent, cow, pig, sheep, horse, goat, dog or cat origin, but any other mammalian cell may be used.

E. coli is one prokaryotic host particularly useful for cloning the polynucleotides of the present invention. Other microbial hosts suitable for use include bacilli, such as Bacillus subtilis, and other enterobacteriaceae, such as Salmonella, Serratia, and various Pseudomonas species.

Other microbes, such as yeast, are also useful for expression. Saccharomyces and Pichia are exemplary yeast hosts, with suitable vectors having expression control sequences (e.g., promoters), an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for methanol, maltose, and galactose utilization.

Further, by use of, for example, the yeast ubiquitin hydrolase system, in vivo synthesis of ubiquitin-transmembrane polypeptide fusion proteins can be accomplished. The fusion proteins so produced can be processed in vivo or purified and processed in vitro, allowing synthesis of polypeptide construct with a specified amino terminus sequence. Moreover, problems associated with retention of initiation codon-derived methionine residues in direct yeast (or bacterial) expression maybe avoided. Sabin et al., 7 Bio/Technol. 705 (1989); Miller et al., 7 Bio/Technol. 698 (1989). Any of a series of yeast gene expression systems incorporating promoter and termination elements from the actively expressed genes coding for glycolytic enzymes produced in large quantities when yeast is grown in mediums rich in glucose can be utilized to polypeptide constructs. Known glycolytic genes can also provide very efficient transcriptional control signals. For example, the promoter and terminator signals of the phosphoglycerate kinase gene can be utilized.

Production of polypeptide constructs in insects can be achieved, for example, by infecting the insect host with a baculovirus engineered to express a transmembrane polypeptide by methods known to those of skill in the art.

In addition to microorganisms, mammalian tissue culture may also be used to express and produce the polypeptide constructs. Expression vectors for these cells can include expression control sequences, such as an origin of replication, a promoter, and an enhancer (Queen et al., Immunol. Rev. 89:49 (1986)), and necessary processing information sites, such as ribosome binding sites, RNA splice sites, polyadenylation sites, and transcriptional terminator sequences. Preferred expression control sequences are promoters derived from immunoglobulin genes, SV40, adenovirus, bovine papilloma virus, cytomegalovirus and the like. See Co et al., J. Immunol. 148:1149 (1992).

The vectors containing the sequences encoding polypeptide constructs of interest can be transferred into the host cell by well-known methods, which vary depending on the type of cellular host. For example, calcium chloride transfection is commonly utilized for prokaryotic cells, whereas calcium phosphate treatment, electroporation, lipofection, biolistics or viral-based transfection may be used for other cellular hosts. (See generally Sambrook et al., Molecular Cloning: A Laboratory Manual (Cold Spring Harbor Press, 2nd ed., 1989). Other methods used to transform mammalian cells include the use of polybrene, protoplast fusion, liposomes, electroporation, and microinjection (see generally, Sambrook et al., supra). For production of transgenic animals, transgenes can be microinjected into fertilized oocytes, or can be incorporated into the genome of embryonic stem cells, and the nuclei of such cells transferred into enucleated oocytes.

Provided herein is a method of producing a polypeptide construct disclosed herein, the method comprising providing a cell expressing a polypeptide construct herein and isolating the polypeptide construct.

Provided herein is a method of producing a polypeptide construct disclosed herein, the method comprising providing a polypeptide according to SEQ ID 21-40 and ligating the polypeptide to a heterologous polypeptide, wherein ligation is carried out by a chemical reaction (such as click chemistry) utilizing a modified amino acid residue at the terminus of the polypeptide.

In one embodiment, some or all the components making up the polypeptide construct are linked by chemical ligation. Click chemistry is one exemplary ligation method; various methods for linking molecules by click chemistry are well known in the art. “Click Chemistry” is a term introduced by researchers at the Scripps Research Institute to describe chemistry tailored to generate substances quickly and reliably by joining small units together. The term “click chemistry” applies to reactions that are highly efficient, wide in scope, and stereospecific. Product isolation is easy, the reactions are simple to perform using inexpensive reagents and can be conducted in benign solvents such as water. The Huisgen 1,3-dipolar cycloaddition is probably the most extensively studied click reaction. A variant of this reaction, the copper-catalyzed azide-alkyne cycloaddition (CuAAC) also fits the click chemistry concept well and is one of the most popular prototype click reactions to date.

Classic click chemistry methods typically rely on a heterobifunctional cross-linker, such as N-hydroxysuccinimide (NHS)-linker-maleimide, or a similar two-step process. It utilizes the amino-reactive NHS and the thiol-reactive maleimide to conjugate protein to the solid support. Other methods include combining a strain-promoted azide-alkyne cycloaddition (SPAAC) click reaction and an OaAEP1 (C247A)-based enzymatic ligation. These methods are well known in the art and can be applied with the present disclosure by one of ordinary skill in the art.

In some embodiments, one or both of the polypeptide, the heterologous polypeptide, and/or the polypeptide construct contains a chemical modification to one or more amino acids, and/or the addition or conjugation of a functional moiety. Such amino acid modifications include, but are not limited to, phosphorylation, methylation (e.g., lysine methylation (mono-, di-, or trimethylation) and arginine methylation (mono, asymmetric dimethylation, or symmetric dimethylation)), acetylation, ubiquitination, myristoylation, palmitoylation, isoprenylation, prenylation, acylation, glycosylation, hydroxylation, iodination, oxidation, sulfation, selenoylation, SUMOylation, citrullination, deamidation, carbamylation, ADP-ribosylation, ubiquitination, nitrosylation, lysine crotonylation, formylation, propionyllysine, butyryllysine, or any combination thereof. In some embodiments, the polypeptide construct is covalently modified with one or more lipids, including, but not limited to, fatty acids, cholesterol, isoprenoids, phospholipids, and diacylglyceryl lipids. In some embodiments, the polypeptide construct is linked to a functional moiety, including, but not limited to, a diagnostic moiety, or a detectable moiety, a moiety useful for ligation or purification, or a targeting moiety. The conjugation to the functional moiety may or may not be at one of the termini of the polypeptide construct. A moiety may have more than one function. In one embodiment, modification includes an alkyl modified peptide on the terminus of a first polypeptide and an azide modified peptide on the second (heterologous) polypeptide, which facilitate the formation of an amide bond between the modified peptides on the first polypeptide and the second polypeptide, in order to generate a polypeptide construct.

Examples of moieties useful for purification include, but are not limited to, Albumin-binding protein (ABP), Alkaline Phosphatase (AP), AU1 epitope, AU5 epitope, Bacteriophage T7 epitope (T7-tag), Bacteriophage V5 epitope (V5-tag), Biotin-carboxy carrier protein (BCCP), Bluetongue virus tag (B-tag), Calmodulin binding peptide (CBP), Chloramphenicol Acetyl Transferase (CAT), Cellulose binding domain (CBP), Chitin binding domain (CBD), Choline-binding domain (CBD), Dihydrofolate reductase (DHFR), E2 epitope, FLAG epitope, Galactose-binding protein (GBP), Green fluorescent protein (GFP), Glu-Glu (EE-tag), Glutathione S-transferase (GST), Human influenza hemagglutinin (HA), HaloTag®, Histidine affinity tag (HAT), Horseradish Peroxidase (HRP), HSV epitope, Ketosteroid isomerase (KSI), KT3 epitope, LacZ, Luciferase, Maltose-binding protein (MBP), Myc epitope, NusA, PDZ domain, PDZ ligand, Polyarginine (Arg-tag), Polyaspartate (Asp-tag), Polycysteine (Cys-tag), Polyhistidine (His-tag), Polyphenylalanine (Phe-tag), Profinity exact, Protein C, S1-tag, S-tag, Streptavadin-binding peptide (SBP), Staphylococcal protein A (Protein A), Staphylococcal protein G (Protein G), Strep-tag, Streptavadin, Small Ubiquitin-like Modifier (SUMO), Tandem Affinity Purification (TAP), T7 epitope, Thioredoxin (Trx), TrpE, Ubiquitin, Universal, and VSV-G.

Examples of detectable moieties include, but are not limited to, fluorescent moieties or labels, imaging agents, radioisotopic moieties, radiopaque moieties, and the like, e.g., detectable labels such as biotin, fluorophores, chromophores, spin resonance probes, or radiolabels. Non-limiting examples of fluorophores include fluorescent dyes (e.g., fluorescein, rhodamine, and the like) and other luminescent molecules (e.g., luminal). A fluorophore may be environmentally-sensitive such that its fluorescence changes if it is located close to one or more residues in the modified protein that undergo structural changes upon binding a substrate (e.g., dansyl probes). Non-limiting examples of radiolabels include small molecules containing atoms with one or more low sensitivity nuclei (13C, 15N, 2H, 125I, 123I, 99Tc, 43K, 52Fe, 67Ga, 68Ga, 111In and the like). Other useful moieties are known in the art.

Provided herein is a polypeptide construct comprising (a) a polypeptide comprising an amino acid sequence that is at least 80% identical to an amino acid sequence selected from any one of SEQ ID NO:1-40 and (b) heterologous polypeptide. In some embodiments, the polypeptide comprises a sequence is at least 80%, at least 85%, at least 90%, at least 95%, at least 96%, at least 97%, at least 98%, or at least 99% identical to any one of SEQ ID NOs: 1-40. In some embodiments the polypeptide comprises any one of SEQ ID NOs: 1-40. In some embodiments the polypeptide comprises a sequence is at least 80%, at least 85%, at least 90%, at least 95%, at least 96%, at least 97%, at least 98%, or at least 99% identical to SEQ ID NO: 1. In some embodiments the polypeptide comprises SEQ ID NO: 1. In embodiments, the polypeptide comprises a N-terminal or a C-terminal lysine. Also contemplated are polypeptides that contain one or more truncations, internal deletions, internal insertions, substitution, or modifications as compared to any of the polypeptide sequences disclosed herein. For example, a person skilled in the art may wish to truncate a polypeptide sequence disclosed herein to alter the stability of the polypeptide or to increase the ease or cost of producing the polypeptide. Truncated polypeptides (from 50 to 30 amino acids, and 30 to 20 amino acids) were tested and truncations of polypeptide according to SEQ ID NO:1 were shown to exhibit the properties of a polypeptide according to SEQ ID NO 1 (full length) for targeted delivery of a heterologous polypeptide.

Provided herein is a polypeptide construct comprising (a) a polypeptide comprising a modified terminal lysine and (b) a heterologous polypeptide, wherein the heterologous polypeptide is linked to the polypeptide via an amide bond formed between the heterologous polypeptide and the modified terminal lysine of the polypeptide.

Provided herein is a polypeptide construct comprising (a) a polypeptide and (b) heterologous polypeptide, wherein the heterologous polypeptide is a therapeutic polypeptide. It should be noted that while therapeutic polypeptides may be used for treatment purposes, the disclosure is not limited to such use, as said polypeptides may also be used for in vitro studies.

In some embodiments, the therapeutic polypeptide is a hormone, interferon, interleukin, growth factor, tumor necrosis factor, thrombolytic, enzyme, antibody, Fc fusion protein, anticoagulant, blood factor, bone morphogenetic protein, engineered protein scaffold.

In some embodiments, the hormone is an erythropoietin. In some embodiments, hormone is human erythropoietin. In one embodiment, the hormone is epoetin. Not-limiting examples of erythropoietins include Epogen® (epoetin-alfa), Procit® (epoetin alfa-epbx), and Retacrit® (epoetin alfa-epbx), and pegylated epoetin. In some embodiments, the hormone is a glucagon-like peptide 1 (GLP-1) or a GLP-1 agonist. Not-limiting examples of GLP-1 agonists include, but are not limited to, Exendin 4, semaglutide (including but not limited to Wegovy® and Ozempic®), liraglutide (including not limited to Victoza®), exenatide (including not limited to Byetta® and Bydureon®), etc. In some embodiments, the hormone is insulin. In some embodiments, the insulin is insulin aspart, insulin lispro, insulin glulisine, insulin detemir, degludec insulin, and glargine insulin.

In some embodiments, the therapeutic polypeptide is somatostatin, a somatostatin analog, glucagon, galsulfase, nesiritide, or taliglucerase alfa. Not-limiting examples of somatostatins include, but are not limited to, Sandostatin® LAR Depot (octreotide acetate), MYCAPSSA® (octreotide). Note, that Mycapassa uses a Transient Permeation Enhancer (TPE®) to transport octreotide from the stomach to the blood stream. TPE® is an oily suspension of octreotide that includes a number of excipients that can transiently alter epithelial barrier integrity by opening of intestinal epithelial tight junctions arising from transcellular perturbation. It is questioned whether Permeation Enhancers (PEs) can cause irreversible epithelial damage and tight junction openings sufficient to permit co-absorption of payloads with bystander pathogens, lipopolysaccharides and its fragment, or exo- and endotoxins that may be associated with sepsis, inflammation and autoimmune conditions. Most PEs seem to cause membrane perturbation to varying extents that is rapidly reversible, and overall evidence of pathogen co-absorption is generally lacking. It is unknown however, whether the intestinal epithelial damage-repair cycle is sustained during repeat-dosing regimens for chronic therapy. The peptide according to SEQ ID Nos 1-40 does not act as a PE but rather causes the protein to be taken into the intestinal cells and exported back out through the other side to the blood avoiding all the issues associated with PEs.

In some embodiments, the therapeutic polypeptide is a polypeptide such as those disclosed in Table 1 or 2, or a variant thereof. A “variant” refers to a polypeptide that comprises one or more alterations when compared to the parental polypeptide, including, but not limited to amino acid additions, substitutions, insertions, deletions, or posttranslational modifications, wherein the variant retains at least 10% of the therapeutic activity of the parental polypeptide. Also provided are heterologous polypeptides that are biosimilar versions of any of the heterologous polypeptides disclosed herein.

In some embodiments, the heterologous polypeptide is a mammalian polypeptide. In some embodiments, the heterologous polypeptide is a human polypeptide.

In some embodiments, provided is a polypeptide construct comprising an amino acid sequence that is at least 80%, at least 85%, at least 90%, at least 95%, at least 96%, at least 97%, at least 98%, or at least 99% identical to an amino acid sequence selected from any one of SEQ ID NOs: 41-50. In some embodiments, provided is a polypeptide construct comprising an amino acid sequence selected from any one of SEQ ID NOs: 41-50.

In some embodiments, provided is a polypeptide construct comprising: (a) a first polypeptide comprising an amino acid sequence that is at least 80%, at least 85%, at least 90%, at least 95%, at least 96%, at least 97%, at least 98%, or at least 99% identical to an amino acid sequence selected from any one of SEQ ID NO: 1-40, 58, 59; (b) a heterologous polypeptide; and optionally, (c) a second polypeptide comprising an amino acid sequence that is at least 80%, at least 85%, at least 90%, at least 95%, at least 96%, at least 97%, at least 98%, or at least 99% identical to an amino acid sequence selected from any one of SEQ ID NO: 1-40. In some embodiments, provided is a polypeptide construct comprising: (a) a first polypeptide comprising any one of SEQ ID NO: 1-40, 58 or 59; (b) a heterologous polypeptide; and optionally (c) a second polypeptide comprising any one of SEQ ID NO: 1-40, 58 or 59.

In some embodiments, provided is a polypeptide construct comprising: (a) a first polypeptide comprising an amino acid sequence that is at least 80%, at least 85%, at least 90%, at least 95%, at least 96%, at least 97%, at least 98%, or at least 99% identical to an amino acid sequence selected from any one of SEQ ID NO: 1-40, 58, and 59; (b) a heterologous polypeptide comprising an amino acid sequence that is at least 80%, at least 85%, at least 90%, at least 95%, at least 96%, at least 97%, at least 98%, or at least 99% identical to any one of SEQ ID Nos 41-50; and optionally, (c) a second polypeptide comprising an amino acid sequence that is at least 80%, at least 85%, at least 90%, at least 95%, at least 96%, at least 97%, at least 98%, or at least 99% identical to an amino acid sequence selected from any one of SEQ ID NO: 1-40. In some embodiments, provided is a polypeptide construct comprising: (a) a first polypeptide comprising any one of SEQ ID NO: 1-40, 58, 59; (b) a heterologous polypeptide.

TABLE 2
Therapeutic Proteins Tested with a polypeptide
according to SEQ ID 1-40; confirmed in vitro
uptake by Caco-2 cells and/or in vivo delivery

Belatacept
C1 Esterase
Elosulfase alfa
GLP-1 receptor agonist (Exenatide)
Erythropoietin
Factor IX
Factor VIII
FGF7
G-csf
Glucarpidase Interleukin-1 Alpha
Liraglutide
Neupogen
Octreotide
Parathryroid hormone full
Peginterferon beta-1a

Also provided herein are nucleic acids, genomes, and vectors comprising nucleic acids encoding polypeptide constructs disclosed herein. The term “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases.

Provided herein are nucleic acids comprising (i) a promoter and (ii) a transgene encoding a polypeptide construct disclosed herein, wherein the transgene is operably linked to the promoter. As used herein, “operably linked” refers to both expression control sequences that are contiguous with the transgene and expression control sequences that act in trans or at a distance to control the expression of the transgene. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., Kozak consensus sequence); sequences that enhance protein stability; and when desired, sequences that enhance protein processing and/or secretion.

In one aspect, provided is a cell comprising a transgene encoding a polypeptide construct disclosed herein. Provided is a method of making a polypeptide construct disclosed herein, the method comprising (i) providing a cell comprising a transgene encoding an IL-polypeptide construct disclosed herein; and (ii) expressing polypeptide construct in the cell. In some embodiments, the polypeptide construct is substantially purified from the cell. In some embodiments, provided is a cell comprising a transgene encoding a polypeptide construct disclosed herein, wherein the cell secretes the polypeptide construct. In some embodiments, the cell is a bacterial cell, a yeast cell, an insect cell, or a mammalian cell. Provided herein is an isolated cell.

Provided herein are pharmaceutical compositions that comprise a polypeptide construct disclosed herein formulated together with one or more pharmaceutically acceptable excipients. The active agent and excipient(s) may be formulated into compositions and dosage forms according to methods known in the art. The pharmaceutical compositions disclosed herein may be specially formulated in solid or liquid form, including those adapted for oral administration.

Therapeutic compositions comprising a polypeptide construct disclosed herein may formulated with one or more pharmaceutically-acceptable excipients, which can be a pharmaceutically-acceptable material, composition or vehicle, such as a liquid or solid filler, diluent, carrier, manufacturing aid (e.g., lubricant, talc magnesium, calcium or zinc stearate, or steric acid), solvent or encapsulating material, involved in carrying or transporting the therapeutic compound for administration to the subject, bulking agent, salt, surfactant and/or a preservative. Some examples of materials which can serve as pharmaceutically-acceptable excipients include: sugars, such as lactose, glucose and sucrose; starches, such as corn starch and potato starch; cellulose and its derivatives, such as sodium carboxymethyl cellulose, ethyl cellulose and cellulose acetate; gelatin; talc; waxes; oils, such as peanut oil, cottonseed oil, safflower oil, sesame oil, olive oil, corn oil and soybean oil; glycols, such as ethylene glycol and propylene glycol; polyols, such as glycerin, sorbitol, mannitol and polyethylene glycol; esters, such as ethyl oleate and ethyl laurate; agar; buffering agents; water; isotonic saline; pH buffered solutions; and other non-toxic compatible substances employed in pharmaceutical formulations.

A bulking agent is a compound which adds mass to a pharmaceutical formulation and contributes to the physical structure of the formulation in lyophilized form. Suitable bulking agents according to the present disclosure include mannitol, glycine, polyethylene glycol and sorbitol.

The use of a surfactant can reduce aggregation of the reconstituted protein and/or reduce the formation of particulates in the reconstituted formulation. The amount of surfactant added is such that it reduces aggregation of the reconstituted protein and minimizes the formation of particulates after reconstitution. Suitable surfactants according to the present disclosure include polysorbates (e.g., polysorbates 20 or 80); poloxamers (e.g., poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; and polyethyl glycol, polypropyl glycol, and copolymers of ethylene and propylene glycol (e.g., Pluronics, PF68, etc.).

Preservatives may be used in formulations disclosed herein. Suitable preservatives for use in the formulations disclosed herein include octadecyldimethylbenzyl ammonium chloride, hexamethonium chloride, benzalkonium chloride (a mixture of alkylbenzyl-dimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. Other suitable excipients can be found in standard pharmaceutical texts, e.g., in “Remington's Pharmaceutical Sciences”, The Science and Practice of Pharmacy, 19th Ed. Mack Publishing Company, Easton, Pa., (1995).

In embodiments, a pharmaceutical composition comprises a polypeptide construct for oral administration, wherein the composition may be in the form of a solid, a semi-solid, a gel or a liquid, including the form of a tablet, a capsule, a lozenge, or an aqueous solution.

Provided herein is a method of transporting a polypeptide construct comprising a heterologous polypeptide from the gastrointestinal tract of a subject in need thereof to the circulatory system of the subject, the method comprising orally administering to the subject a polypeptide construct disclosed herein or a pharmaceutical composition comprising a polypeptide construct disclosed herein. Provided herein is a polypeptide construct comprising a heterologous polypeptide for use in a method of transporting the polypeptide construct from the gastrointestinal tract of a subject in need thereof to the circulatory system of the subject, the method comprising orally administering to the subject a polypeptide construct disclosed herein or a pharmaceutical composition comprising a polypeptide construct disclosed herein. In embodiments, the subject is a mammal. In embodiments, the subject is a human.

In embodiments, the polypeptide construct comprising a polypeptide and a heterologous polypeptide is absorbed by the apical cell wall of the intestine, travels and exits through the basal wall into the circulatory system. The circulatory system includes the heart, blood vessels (including arteries, veins, capillaries), and blood. In embodiments, the polypeptide construct is absorbed by the stomach walls. In embodiments, the heterologous polypeptide is separated from the remainder of the polypeptide construct once the heterologous polypeptide is located in the circulatory system. In one embodiment, after separation from the remainder of the polypeptide construct, the heterologous polypeptide comprises an N-terminal or C-terminal adduct derived from the from the remainder of the polypeptide construct. In embodiments, the N-terminal or C-terminal adduct is selected from A, GA, RGA, GRGA, or a combination thereof.

Provided herein are methods in which a polypeptide construct disclosed herein is administered to a subject in a therapeutically effective amount.

Disclosed is a method for translocating a therapeutic polypeptide across the gastrointestinal lining of a subject to the circulatory system of the subject, which comprises ingesting a pharmaceutical composition comprising a polypeptide construct comprising a first polypeptide comprising a polypeptide having a sequence identity selected from SEQ ID 1-40 linked to a heterologous polypeptide, wherein the heterologous polypeptide is a therapeutic polypeptide, and wherein the subject is a human, wherein the therapeutic polypeptide is cleaved in whole (or in part) from the first polypeptide prior to (or at the time) the therapeutic polypeptide enters the circulatory system, further wherein the therapeutic polypeptide present in the circulatory system optionally comprises an N-terminal adduct or a C-terminal adduct derived from the first polypeptide selected from A, GA, RGA, GRGA, or a combination thereof.

Provided herein is a method of treating anemia in a subject in need thereof, the method comprising administering to the subject a polypeptide construct comprising erythropoietin. Provided herein is a polypeptide construct comprising erythropoietin. Provided herein is the use of a polypeptide construct comprising erythropoietin in the manufacture of a medicament for treating anemia. In embodiments, the erythropoietin is epoetin alfa or a pegylated epoetin. In embodiments, the polypeptide construct comprises SEQ ID NO: 41.

Provided herein is a method of treating diabetes in a subject in need thereof, the method comprising administering to the subject a composition comprising a polypeptide construct comprising a polypeptide with a sequence identity according to SEQ ID NOs: 1-40, and a heterologous polypeptide, the heterologous polypeptide selected from the group consisting of liraglutide, semaglutide, octreotide, GLP-1, insulin, or variants or analogues thereof. Provided herein is the use of the polypeptide construct comprising one of liraglutide, semaglutide, octreotide, GLP-1, insulin, in the manufacture of a medicament for treating diabetes. In embodiments, the polypeptide construct comprises SEQ ID NOs: 42-50.

The polypeptide constructs provided herein can be administered orally. In embodiments, a polypeptide construct is administered one, twice, three times, four times, five times, for six times a day. The polypeptide construct may be administered every other day, three times/week, twice/week, once a week, every two weeks, every three weeks, once a month, once every 8 weeks (or once every 2 months), once every 12 weeks (or once every 3 months), or once every 24 weeks (once every 6 months). The polypeptide construct may be administered over a period of about 1 week to about 2 weeks, about 2 weeks to about 3 weeks, about 3 weeks to about 4 weeks, about 4 weeks to about 5 weeks, about 6 weeks to about 7 weeks, about 7 weeks to about 8 weeks, about 8 weeks to about 9 weeks, about 9 weeks to about 10 weeks, about 10 weeks to about 11 weeks, about 11 weeks to about 12 weeks, about 12 weeks to about 24 weeks, about 24 weeks to about 48 weeks, about 48 weeks or about 52 weeks, or longer.

An effective amount, or therapeutically effective amount, as the case may be, of the polypeptide construct disclosed herein can be determined by methods known in the art. For example, the appropriate dose of a polypeptide disclosed herein may depend on the route of administration and may depend on the subject being treated as well as the severity of the condition to be treated. Using scaling methods, such as allometric scaling, it is possible to predict suitable and exemplary dosage ranges for the administration of compositions, as disclosed herein, to adult humans. Dose scaling is an empirical approach, is well characterized and understood in the art. This approach assumes that there are some unique characteristics on anatomical, physiological, and biochemical process among species, and the possible difference in pharmacokinetics/physiological time is, as such, accounted for by scaling.

In one aspect, provided herein is a composition comprising a polypeptide construct, further comprising an additional therapeutic agent. Such additional agents include, but are not limited to anti-bacterial agents, cytotoxic agents, chemotherapeutic agents, growth inhibitory agents, anti-inflammatory agents, anti-cancer agents, anti-neurodegenerative agents, and anti-infective agents. Agents that are used in such combination therapies may fall into one or more of the preceding categories. The administration of the polypeptide construct and the additional therapeutic agent may be concurrent or consecutive. The administration of the polypeptide construct and the additional therapeutic agent may be separately or as a mixture.

Provided herein is an overview of a targeted drug delivery system for delivery of a composition comprising a polypeptide construct comprising (a) a first polypeptide comprising an amino acid sequence that is at least 80% identical to an amino acid sequence selected from any one of SEQ ID NO: 1-40 (also referenced as “Peptide Transporter”); and (b) a second polypeptide (also referenced as “Therapeutic (Protein)”), wherein the second polypeptide is a therapeutic polypeptide that is heterologous to the first polypeptide, wherein delivery is across the gastrointestinal lining into the bloodstream following oral administration to a subject. Through an active endocytosis process the polypeptide construct is absorbed into the apical cell wall, travels, and exits through the basal wall where the first polypeptide is naturally cleaved off by thrombin in the blood, thereby delivering the therapeutic polypeptide into the blood stream.

Provided herein is an overview of a targeted delivery polypeptide construct and targeted delivery system, wherein a polypeptide is linked to a therapeutic polypeptide, such as a biologic, wherein linkage is via ligation of the polypeptide to the protein.

To facilitate a better understanding of the present disclosure, the following examples of specific embodiments are given. The following examples should not be read to limit or define the entire scope of the disclosure; the examples are offered by way of illustration and not by way of limitation with respect to subject matter claimed herein.

### Example 1

To assess the effectiveness of a polypeptide comprising an amino acid sequence with a sequence identify according to SEQ IDs 1-40 to facilitate targeted delivery of a therapeutic polypeptide, a series of peptide conjugates were constructed comprising of a polypeptide according to SEQ ID 1-40 linked to a heterologous polypeptide comprising a therapeutic polypeptide. Polypeptide conjugates may be studied in vitro using Caco-2 cells, to assess uptake and bioactivity of the therapeutic polypeptide.

The human epithelial cell line Caco-2 (available from Sigma Aldrich) has been widely used as a model of the intestinal epithelial barrier. Originally derived from a colon carcinoma, one of the cell line's most advantageous properties is its ability to spontaneously differentiate into a monolayer of cells with many properties typical of absorptive enterocytes with brush border layer, as found in the small intestine. To mimic the steric conditions in the intestine in vivo, Caco-2 cells may be cultured on permeable filter inserts (such as available from Becton Dickenson, Corning, Costar). Cultivation of Caco-2 cells on filter supports improves the cell's morphological and functional differentiation. It has been well documented that polarized Caco-2 monolayers represent a reliable correlate for studies on the absorption of drugs and other compounds after oral intake in humans. Several studies have compared Caco-2 permeability coefficients with absorption data in humans and found high correlation, particularly if the compounds are transported by passive paracellular transport mechanisms (Lea T. Caco-2 Cell Line. In: Verhoeckx K, Cotter P, López-Expósito I, et al., editors. The Impact of Food Bioactives on Health: in vitro and ex vivo models [Internet]. Cham (CH): Springer; 2015. Chapter 10. Available from: https://www.ncbi.nlm.nih.gov/books/NBK500149/doi: 10.1007/978-3-319-16104-4_10).

Various polypeptide constructs were labeled with Alexa Fluor™ Labeling Kits (ThermoFisher), to generate fluorescently labeled polypeptide constructs. Caco-2 cells were seeded in 12-well plates and incubated for 4 hours with the labeled polypeptides at concentrations ranging from 2-10 ug/mL. Following incubation, the plates were read on a Tecan Infinite M Nano+ and washed gently several times with PBS, then given fresh media, and the plates were re-read. The % of fluorescence remaining in the dish represents the relative amount of uptake of polypeptide construct compared to the amount originally applied to the cells. (See FIG. 2) Uptake by Caco-2 cells of polypeptide constructs suggests that the constructs are suitable delivery agents for therapeutic polypeptides across the gastrointestinal lining, with the polypeptide according to SEQ ID Nos 1-40 acting as a “shuttle” to transport the therapeutic peptide to which it is linked, from the gut to the bloodstream, thus the Caco-2 cell model assay is a screening tool for various polypeptide therapeutics, to confirm their suitability as an oral dose formulation.

### Example 2

Human erythropoietin (Epo) is a 30.4 kDa glycoprotein hormone composed of a single 165 amino acid residues chain to which four glycans are attached. Epo is the key element in the feedback control of the production of red blood cells in bone marrow. Epoetin is a recombinant form of human erythropoietin (composed of 166 amino acid residues chain) and is used to increase differentiation of progenitor cells to red blood cells in the treatment of anemia (amongst other treatments). Epoetin alfa, branded as Epogen®, is a synthetic protein that helps the body produce red blood cells, primarily used to treat anemia. Epogen® (like most large molecule therapeutics) must be administered intravenously because it cannot be absorbed in the gut. These properties made human erythropoietin a useful test molecule for an oral formulation composition using the methods disclosed herein. (See FIG. 3) A polypeptide construct was constructed comprised of a polypeptide with a sequence identity according to SEQ ID 1 linked to the amino terminal of the Human erythropoietin (“Epo”) sequence. The linkage resulted in a chimeric protein comprising a polypeptide with an amino acid sequence identity corresponding to SEQ ID 41.

The polypeptide according to SEQ ID 41 was cloned out by inserting the (human) erythropoietin sequence, upstream of SEQ ID 1, into a pBluescript bacterial expression vector (Sigma Aldrich). This bacterial construct culture was grown under standard culture conditions to express the polypeptide according to SEQ ID 41. The bacteria slurry containing the polypeptide according to SEQ ID 41 was centrifuged and re-suspended in 25 ml column buffer (CB) per liter of culture. The bacteria cells were lysed by freeze-thaw followed by passaging through a 20-gauge needle. The lysed cells were centrifuged, and the supernatant was diluted by adding 125 ml cold CB for every 25 ml crude extract. The diluted crude extract was added to a Protein A microbead column containing antibodies to human erythropoietin and was washed with 12 column volumes of CB, and after washing was eluted with elution buffer. The amount of isolated polypeptide according to SEQ ID 41 was determined by bicinchoninic acid assay (BSA) assay and the amount of protein was measured via ELISA, and the purity was assessed with the ratio of protein-to-SEQ ID 41 protein in the eluant.

Eight (8) 12-week-old male Wistar rats were administered by PO a composition comprising a polypeptide according to SEQ ID 41 (400 μg in 200 μL PBS) or were administered by PO a control (200 μL PBS only). Administration was PO once a day for four weeks. Blood was drawn via tail vein from all treated rats at 0, 14 and 28 days. (See FIG. 7)

After 2 weeks of treatment, a fragment of polypeptide according to SEQ ID 56 was detected in the blood of 7 of the 8 animals (a range of 0.8-23 ng/ml) and in all of the animals by week 4 (1.2-22 ng/mL), indicating the polypeptide construct was able to travel across the gastrointestinal lining, allowing the therapeutic to cross from the gastrointestinal (GI) tract into the blood stream following oral administration.

To assess if the composition comprising a polypeptide construct according to SEQ ID 41 was biologically active following oral administration, the hemoglobulin levels of the animals were measured post-treatment. After 2 weeks of treatment the average hemoglobulin level increased from 8.79 gm/dl to 9.211 gm/dl, while controls remained relatively constant at 8.87 gm/dl and 8.84 gm/dl, respectively. By the end of the treatment (4 weeks) the controls remained below 9 with an average reading of 8.96 gm/dl while the controls increased further to 9.64 gm/dl. Hence, the composition comprising a polypeptide construct comprising an amino acid sequence according to SEQ ID 41, when administered orally, not only crossed the gastrointestinal lining of the GI tract and entered the bloodstream of the animals but remained biologically and therapeutically active following uptake to the bloodstream.

### Example 3

Four (4) Sprague Dawley rats were administered a composition comprising a polypeptide according to SEQ ID 41 at a dosage of 600 μg, followed by exsanguination at 4 (n=2) and 6 hours (n=2) post-treatment. (See FIG. 6) Blood from the animals was then processed for removal of most blood proteins, which were size selected on a Western Blot, followed by peptide exclusion via PAGE. From the electrophoresis gel a band between 15-25k DA was excised and sequenced. The resulting sequence of the serum derived peptide was determined to be a polypeptide according to SEQ ID 56 (corresponding to the erythropoietin sequence (SEQ ID 51), with the addition of two amino acids (GA) at the amino end, which indicates that 48 of the 50 amino acids of SEQ ID 1 were cleaved from SEQ ID 41 following administration.

To assess bioavailability, ten (10) Sprague Dawley rats were administered a composition comprising a polypeptide according to SEQ ID 41 via oral gavage at concentrations of 2.5 mg/kg (n=5) or 1 mg/kg (n=5). Following administration, blood was drawn from each animal at 0, 5, 15, 30, 60 minutes, and 2-, 4-, 8- and 24-hours post-treatment. The amount of the composition comprising a polypeptide according to SEQ ID 41, or fragment thereof, in the blood was measured and compared to values from animals treated with similar composition by intravenous injection, at a dose of 0.5 mg/kg (n=5). Bioavailability (F) is the fraction (%) of an administered drug that reaches the systemic circulation. Mathematically, bioavailability equals the ratio of comparing the area under the plasma drug concentration curve versus time (AUC) for the extravascular formulation to the AUC for the intravascular formulation. AUC is utilized because AUC is proportional to the dose that has entered the systemic circulation.

In order to determine absolute bioavailability of a drug, a plasma drug concentration versus time plot for the drug after both intravenous (iv) and extravascular (non-intravenous, i.e., oral) administration was determined. The absolute bioavailability is the dose-corrected area under curve (AUC) non-intravenous divided by AUC intravenous. Therefore, a drug given by the intravenous route will have an absolute bioavailability of 100% (f=1), whereas drugs given by other routes usually have an absolute bioavailability of less than one.

Following this concentration v. time plot/AUC model, it was determined that a polypeptide construct comprising an amino acid sequence identity according to SEQ ID 41 had an average F-value of 0.18 with a range from 0.062 to 0.32. The maximum (or peak) serum concentration (Cₘₐₓ) achieved was an average Cₘₐₓ of 202.6 mlUnits/ml, with an average tₘₐₓ (time to reach maximum concentration) of 180 minutes. Every animal that received an oral dose of a composition comprising a polypeptide according to SEQ ID 41 achieved levels corresponding to therapeutic levels of serum Epogen®.

### Example 4

Sprague Dawley rats were administered a composition comprising SEQ ID 41 (doses of 2.5 mg/kg n=5, 1 mg/kg n=5 and 0.25 mg/kg n=5) by PO and also by IV (a dose of 0.5 mg/kg n=5), blood was drawn at 0, 5, 15, 30, 60 minutes, and 2, 4, 8 and 24 hours post treatment systemic levels of the peptide were compared between PO and IV treated animals. In the PO treated animals, a peptide according to SEQ ID 42 was detected in the blood as early as 30 minutes' post-administration, with a peak level between 4 and 6 hours, an F value of 0.3, and circulating levels between 150 and 240 mlU/mL with sustained levels for 24 hours post administration. (See FIG. 4A)

To test the hypothesis that a polypeptide comprising SEQ ID 1, or fragment thereof, is cleaved from the peptide construct comprising SEQ ID 41 upon uptake into the bloodstream, an ELISA assay was carried out to detect the 50-aa PT sequence in the blood, post administration. As was seen with the detection of Epo, a polypeptide according to SEQ ID NO:57 could be detected as early as 30 minutes' post administration, and levels peaked at 4 hours. However, unlike Epo levels, the concentration of the polypeptide according to SEQ ID NO:57 decreased to less than 40% within 8 hours post administration, and became non-detectable within 24 hours, confirming cleavage and degradation of the polypeptide. (See FIG. 5)

To determine the efficacy of a composition comprising a polypeptide construct comprising an amino acid sequence identity according to SEQ ID NO:41 (referred to as “PT-EPO”), Sprague Dawley rats (n=8) were given PT-EPO daily (PO) at 2 mg/kg for 28 days. Blood was drawn on days 0, 14 and 28 and Epo and hemoglobin levels were measured. After two weeks of PT-EPO treatment, an average of 10.1125 pg/mL Epo could be detected in the animals' serum and this level increased to 11.425 pg/mL by day 28. (See FIG. 7) Treatment with PT-EPO was also associated to a significant increase in hemoglobin levels, from 16.35 ng/ml starting, to 17.1375 ng/ml on day 14 and 17.9375 ng/ml on day 28 (p<0.05), while control animals showed no significant changes. (See FIG. 8)

### Example 5

In a 14-day safety study in a canine animal model (beagles) bioavailability of an orally administered composition comprising a polypeptide construct comprising an amino acid sequence having a sequence identity according to SEQ ID 41 (PT-EPO) was assessed. Animals were administered (PO) a composition comprising PT-EPO at daily doses of 0, 5, 50 and 125 mg/kg for 14 consecutive days. Following termination of the study, the animals underwent pathological analysis, and it was found that PT-EPO had no negative effects on any major organ function. The safety study also demonstrated that PT-EPO had no deleterious effects when administered to dogs daily, over 14 days, even at high doses (125 mg/kg). Furthermore, after 14 days of administration (PO) of a composition comprising PT-EPO, the treated animals had increased levels of red blood cells (6.74 vs 7.26×10⁶ cells per ul), hemoglobin (15.85 vs 17.125 g/dl) and hematocrit (46.8 vs 49.65%) compared to controls, therefore confirming bioavailability of the orally administered composition comprising a polypeptide construct.

### Example 6

GLP-1 agonists, such as exenatide and liraglutide are desirable candidates for formulation as an oral dose therapeutic. To test if a polypeptide according to SEQ ID 1-40, when linked to a GLP-1 agonist as a polypeptide construct, facilitates the transport of GLP-1 agonists from the stomach to the blood, following oral administration. The amino acid sequences for exenatide and liraglutide were used to clone the sequences behind (downstream) of a polypeptide comprising an amino acid sequence according to SEQ ID NO 1, in order to generate, using an expression vector system, peptide constructs comprising an amino acid sequence according to SEQ ID NOs: 42,44 (referred to in the figures as “PT-GA1” and “PT-GA2”, respectively). Also, generated was a polypeptide construct according to SEQ ID NO:46 (“PT-GA2B”), comprised of the liraglutide sequence flanked by a polypeptide according to SEQ ID NO 1 on both the carboxy and amino terminal end of the liraglutide sequence. The polypeptide constructs were tested in the Caco-2 uptake assay (Example 1), and it was determined that PT-GA1 had an update of 28.7%, PT-GA2 of 31.4% and PT-GA2B of 29.4%. (See FIG. 11) To test efficacy and bioavailability in vivo, Sprague Dawley rats were administered (PO) 600 ug of the compositions comprising polypeptide constructs selected from PT-GA1, PT-GA2 or PT-GA2B, blood was drawn at 0, 4, and 6 hrs. post-treatment, and blood glucose levels were measured. Following treatment, the blood glucose levels of the PT-GA1 treated animals went from 107 mg/dl at 0 hrs to 118 mg/dL at 4 hrs and dropped to 87.5 mg/dl at 6 hrs. post treatment, while the PT-GA2 and PT-GA2B were 98 mg/dl, 126, 111 mg/dL; and 102 mg/dL, 105 mg/dL, 104 mg/dl respectively.

### Example 7

An alternate strategy for generating peptide constructs disclosed herein includes chemical synthesis and ligation. In one example, a polypeptide according to SEQ ID 21-40 with a modified terminal residue may be chemically ligated to a heterologous polypeptide with a modified terminal residue. For example: a terminal lysine of the polypeptide may be an alkyl modified peptide; and a terminal residue of the heterologous polypeptide may be an azide modified peptide, wherein the alkyl modified peptide reacts with the azide modified peptide to generate an amide bond between the polypeptide and the heterologous polypeptide.

In one embodiment, the terminal amino acid of a peptide according to SEQ ID 1-20 may include a modified lysine residue: Lys(N3)=Fmoc-L-Lys(N3)-OH. In one exemplary formula, a modified peptide according to SEQ ID 21 comprises a formula according to:

H-Met-Ala-Asp-Asp-Ala⁵-Gly-Ala-Ala-Gly-Gly¹⁰-Pro-Gly-Gly-Pro-Gly¹⁵-Gly-Pro-Gly-Met-Gly²⁰-Asn-Arg-Gly-Gly-Phe²⁵-Arg-Gly-Gly-Phe-Gly³⁰-Ser-Gly-Ile-Arg-Gly³⁵-Arg-Gly-Arg-Gly-Arg⁴⁰-Gly-Arg-Gly-Arg-Gly⁴⁵-Arg-Gly-Arg-Gly-Lys(N3)⁵⁰-OH; wherein the polypeptide is linked, via the Lysine terminal residue, to a heterologous polypeptide comprising a therapeutic polypeptide, wherein the heterologous polypeptide is a modified polypeptide according to the formula: Propynoic Acid-D-Phe-Cys-Phe-D-Trp-Lys-Thr-Cys-Thr-ol.

In one embodiment, a copper (I)-catalyzed alkyne azide 1,3-dipolar cycloaddition (CuAAC) or ‘click’ reaction, is utilized to link a peptide according to SEQ ID 21-40 to a heterologous polypeptide. A copper-catalyzed click reaction is a highly versatile reaction that can be performed under a variety of reaction conditions including various solvents, a wide pH and temperature range, and using different copper sources, with or without additional ligands or reducing agents. This reaction is highly selective and can be performed in the presence of other functional moieties. The 1,4-disubstituted triazole product of the CuAAC reaction is a suitable isostere for an amide bond.

In one example, a heterologous polypeptide is an octapeptide, which mimics natural somatostatin's pharmacological effect (analogous to Octreotide, branded as Sondostatin®), and may be ligated to a polypeptide according to SEQ ID 21-40 (modified) to generate a polypeptide construct according to SEQ ID 50 (designated herein as “PT-OCT” or “PT-OCT click”).

A compound generated by ligation, comprises the formula H-Met-Ala-Asp-Asp-Ala-Gly-Ala-Ala-Gly-Gly-Pro-Gly-Gly-Pro-Gly-Gly-Pro-Gly-Met-Gly-Asn-Arg-Gly-Gly-Phe-Arg-Gly-Gly-Phe-Gly-Ser-Gly-Ile-Arg-Gly-Arg-Gly-Arg-Gly-Arg-Gly-Arg-Gly-Arg-Gly-Arg-Gly-Arg-Gly-Nle (triazol-propionyl-D-Phe-Cys-Phe-D-Tru-Lys-Thr-Cys-Thr-ol)-OH

The compound having the following characteristics:
Appearance: White to off-white powder.
Identity: Mass spectrometry M.W. (average)=5822.5+Iamu; (M+4H) 4+/4=1456.9/(M+4H) 5+/5=1165.7 After deconvolution: M.W.=5823.6 amu.
Purity: RP-HPLC 90%.
Net Peptide Content (NPC) (Nitrogen analysis): 83.5%.
Water Content (Karl Fischer USP <921> 5.2%; and
Total Mass Balance (NPC+Counter Ion (Acetate): 90-105%/97%.

Polypeptide constructs generated by ligation methods, such as click chemistry, can also be tested using in vitro methods described herein, including a Caco-2 uptake assay to access the uptake of the polypeptide constructs into Caco-2 cells, which mimics uptake of the polypeptide constructs across the gastrointestinal barrier. (See Example 1)

Also generated was a polypeptide construct comprising a polypeptide having an amino acid sequence identity according to SEQ ID NO 49 (utilizing an expression vector method as disclosed herein and referred to as “PT-OCT fusion”); comprising a polypeptide having an amino acid sequence identity according to SEQ ID NO 50 (utilizing a chemical “click” ligation method and referred to as “PT-OCT click”) and the two were compared side-by-side in vivo and in vitro. PT-OCT fusion and PT-OCT click constructs were shown to be effective at causing uptake in vitro (in Caco-2 cells). In one example, approximately 38% of the polypeptide constructs (PT-OCT fusion and PT-OCT click) administered to Caco-2 cells were taken up by the Caco-2 cells. (Uptake by PT-EPO was also carried out as a reference)

To determine if the polypeptide constructs comprising a polypeptide having an amino acid sequence identity according to SEQ ID NO:49 or 50 are biologically active following administration (PO), the ability of PCT-OCT to inhibit insulin secretion from glucose-stimulated islets was tested. Triplicates of 25 human islets (from a donor pancreas procured from an IIDP-approved islet center) were plated in a 96-well plate containing 100 μl of CMRL islet media (such as available from MediaTech). The islets were pre-incubated with either 100 μl (50%) serum (serum was collected from pancreas donor), 100 μl (50%) serum+PT-OCT, or 100 μl (50%) serum+PT-OCT that was exposed to thrombin for 45 minutes to cleave the polypeptide sequence from the therapeutic octapeptide of the polypeptide construct. Following a pre-incubation, the media and serum was removed and replaced with KREB's buffer solution containing 16.7 mM glucose, and incubated for an additional 30 minutes, at which point the KREB's buffer was collected. Insulin concentrations of the supernatant was measured using commercially available ELISA kits (such as available from Abcam). Both polypeptide constructs (PT-OCT and PCT-OCT “cut”) exhibited the ability to inhibit the insulin secretion of glucose-stimulated islets, with the polypeptide construct comprising full length PT-OCT decreasing insulin secretion by 46%, while the polypeptide construct comprising PCT-OCT “cut” that was cut by thrombin, inhibited the expression of insulin in glucose-stimulated islets by over 61%. (See FIG. 19)

### Example 8

The following paragraphs describe additional data:

Provided herein is an illustration of the uptake and bioavailability of a targeted delivery method for oral administration of erythropoietin (See Examples 2-5). Human erythropoietin with N-terminal glycine and alanine resides was separated from other serum proteins using Western Blot. The band was sequenced and confirmed to be an amino acid sequence corresponding to the full-length human erythropoietin sequence, with two additional amino acid residues (“GA”) remaining. This data confirms that a polypeptide construct of SEQ ID 41 crossed the intestinal barrier of the gastrointestinal (GI) tract when administered orally and that a polypeptide of SEQ ID 56 was taken up into the bloodstream.

Provided herein is a demonstration of the therapeutic efficacy in a second animal (canine) model following oral administration of a composition comprising a polypeptide construct with a sequence identity corresponding to SEQ ID 41 (See Example 5). The hemoglobin and hematocrit levels increased in the subject dogs (beagles) following oral administration of a composition comprising a polypeptide with a sequence identity corresponding to SEQ ID 41. Doses administered were 1 mg/kg, 5 mg/kg, 50 mg/kg, 125 mg/kg, each in PBS and administered PO as a single dose.

We have obtained the aggregate data of red blood cell counts, hemoglobin levels, and hematocrit levels following administration PO of a single dose of a composition comprising a polypeptide construct according to SEQ ID 41, compared to control (data not shown, see Example 5).

Provided herein is a demonstration of the therapeutic efficacy in vivo of a composition comprising a polypeptide construct according to SEQ ID 42 or 44, as seen by a decrease in blood glucose levels following oral administration of the composition (dosage 600 μg in PBS).

Provided herein is a polypeptide with a sequence identity according to SEQ ID 21 with a modified moiety at the C-terminal end comprising of modified Lysine residue, (Lys(N3), which is a ligand for ligation and linking of the polypeptide to a second heterologous polypeptide. Also provided is a heterologous polypeptide comprising an octreotide analogue (Pentynoyl-Octreotide, as modified) for ligation (linking) to a polypeptide having a sequence identity according to SEQ ID 21-40 (See Example 7).

Further provided herein is a formula comprising a polypeptide construct (designated “PT-OCT”) comprised of a polypeptide according to SEQ ID 21 ligated, via click chemistry, to a heterologous polypeptide, wherein the heterologous polypeptide is an octapeptide (Octreotide analogue) according to SEQ ID 53 (and modified according to the Pentynoyl-Octreotide provided herein).

Provided herein is a demonstration of the uptake in vitro of polypeptide constructs with a sequence identity corresponding to SEQ ID Nos: 41, 49 and 50. Caco-2 cells were treated with a composition comprising a polypeptide construct with a sequence identity corresponding to SEQ ID 41 (also referenced as PT-EPO), a polypeptide construct with a sequence identity corresponding to SEQ ID 49 (also referenced as PT-OCT “Fusion”) and a polypeptide construct with a sequence identity corresponding to SEQ ID 50 (also referenced as PT-OCT “Ligated”) each at a concentration of 5 μg/mL for 2 hours. Based on a fluorescent assay it was determined that over 30% of the constructs were taken up by the Caco-2 cells, indicating the ability of the composition to traverse the gastrointestinal tract. The side-by-side comparison also validates the use of click chemistry ligation as a method of generating polypeptide constructs as disclosed herein, along with traditional expression vector and other recombinant methods of generating polypeptide constructs.

Provided herein is a demonstration of the therapeutic efficacy of a peptide construct comprising a polypeptide with a sequence identity according to SEQ ID 50 (also referenced as “PT-OCT”) and the same construct following cleavage by thrombin (also referenced as “PT-OCT (cut)”). Each of the polypeptide constructs (full length and “cut”) cause a reduction in relative glucose secretion by glucose stimulated islet cells (compared to control), thus confirming the ability of the polypeptide constructs to inhibit insulin secretion of glucose-stimulated islet cells.

The foregoing Examples are supplemented by the accompanying Figures and by Table 1 and Table 2. Some of the Examples and Figures refer to compositions and constructs of the present disclosure other than by sequence number, nomenclature of which is described in Table 1.

Polypeptides according to SEQ ID 1-40 have been shown to be safe and effective for targeted drug delivery. In general, the use of peptides in pharmaceutical formulations are considered to be rapidly cleaved by proteolytic enzymes and quickly cleared from the blood circulation by liver and kidney; these pharmacodynamic properties can be modulated by different modification and stabilization approaches (Vlieghe et al., 2010). One of the most known concepts of peptide stabilization is lipidation, which involves the incorporation of fatty acids into the peptide (Zhang and Bulaj, 2012). Lipidation of polypeptide constructs disclosed herein are also envisioned by the disclosure. Fatty acids bind to serum albumin, preventing proteolytic cleavage in the blood by proteases and leads thereby to a prolonged circulation time (Frokjaer and Otzen, 2005). The long-acting glucagon-like peptide-1 (GLP-1) receptor agonists liraglutide (Victoza®) (Guryanov et al., 2016) and semaglutide (Ozempic®) (Marso et al., 2016), which are used to treat type-2 diabetes and obesity, are examples for this approach. Peptides are generally considered safe, since they feature low immunogenicity and produce non-toxic metabolites (Ahrens et al., 2012).

Although the foregoing information highlights aspects disclosed herein by way of illustration and example for purposes of clarity of understanding, it will be obvious that certain changes and modifications may be practiced within the scope of the subject matter claimed herein. It will be clear to a person skilled in the art that features described in relation to any of the aspects and various embodiments described above can be applicable interchangeably between the different embodiments.

The aspects and embodiments described above are examples to illustrate various features of the subject matter claimed herein. All publications and patent applications disclosed herein are indicative of the level of those skilled in the art to which this disclosure and the subject matter of the claims pertains.

It will be understood that a numerical value may be associated with a certain amount of experimental error. Thus, recitation of the qualifier “about” (or “approximately”) prior to a numerical error is meant to embody the experimental error that may be associated with the recited numerical value. To the extent that a numerical value obtained experimentally is not preceded by the expression “about” (or “approximately”) does not mean that the numerical value is not associated with a certain amount of experimental error.

Throughout the description and claims of this specification, the words “comprise” and “contain” and variations of them mean “including but not limited to”, and they are not intended to (and do not) exclude other moieties, additives, components, or steps. Throughout the description and claims of this specification, the singular encompasses the plural unless the context otherwise requires. Where the indefinite article is used, the specification is to be understood as contemplating plurality as well as singularity, unless the context requires otherwise.

Features, characteristics, compounds, chemical moieties, or groups described in conjunction with a particular aspect, embodiment, or example are to be understood to be applicable to any other aspect, embodiment or example described herein unless incompatible therewith. All the features disclosed in this specification (including any accompanying claims, abstract and drawings), and/or all the steps of any method or process so disclosed, may be combined in any combination, except combinations where at least some of such features and/or steps are mutually exclusive. The subject matter claimed herein is not restricted to the details of any foregoing embodiments. The subject matter claimed herein extends to any novel one, or any novel combination, of the features disclosed in this specification (including any accompanying claims, abstract and drawings), or to any novel one, or any novel combination, of the steps of any method or process so disclosed.

All publications and patent applications are herein incorporated by reference to the same extent as if each individual publication or patent application was specifically and individually to be incorporated by reference.

1. A polypeptide construct comprising:
(a) a first polypeptide comprising an amino acid sequence that is at least 95% identical to an amino acid sequence selected from any one of SEQ ID NO: 1-40; and
(b) a second polypeptide, wherein the second polypeptide is heterologous to the first polypeptide.

2-7.

8. The polypeptide construct of claim 1, wherein the first polypeptide and the second polypeptide are linked through a linker.

9. (canceled)

10. The polypeptide construct of claim 8, wherein:
(a) the N-terminus of the second polypeptide is linked to the C-terminus of the first polypeptide; or
(b) the N-terminus of the first polypeptide is linked to the C-terminus of the second polypeptide.

11-29. (canceled)

30. The polypeptide construct of claim 1, wherein the second polypeptide is or comprises a therapeutic protein.

31. The polypeptide construct of claim 30, wherein the therapeutic protein is a hormone, interferon, interleukin, growth factor, tumor necrosis factor, thrombolytic, enzyme, antibody, Fc fusion protein, anticoagulant, blood factor, bone morphogenetic protein, engineered protein scaffold.

32. The polypeptide construct of claim 31, wherein the hormone is an erythropoietin.

33. The polypeptide construct of claim 32, wherein the erythropoietin is epoetin alfa or a pegylated epoetin.

34. The polypeptide construct of claim 31, wherein the hormone is a glucagon-like peptide 1 (GLP-1) agonist.

35. The polypeptide construct of 34, wherein the GLP-1 agonist is semaglutide, exenatide, liraglutide.

36. The polypeptide construct of claim 31, wherein the hormone is insulin.

37. The polypeptide construct of claim 36, wherein the insulin is insulin aspart, insulin lispro, insulin glulisine, insulin detemir, degludec insulin, or glargine insulin.

38. The polypeptide construct of claim 30, wherein the therapeutic protein is somatostatin, a somatostatin analog, glucagon, galsulfase, nesiritide, or taliglucerase alfa.

39. The polypeptide construct of any one of the preceding claims, wherein the polypeptide construct comprises an amino acid sequence that is at least 80% identical to any one of SEQ ID NOs: 41, 44-52.

40. The polypeptide construct of claim 30, wherein the polypeptide construct comprises an amino acid sequence that is at least 95% identical to any one of SEQ ID NOs: 41, 44-52.

41-52. (canceled)

52. A pharmaceutical composition comprising the polypeptide construct of claim 1 or claim 30 and optionally a pharmaceutically acceptable excipient.

53. The pharmaceutical composition of claim 52, wherein the pharmaceutical composition further comprises an additive, a stabilizer, a permeability enhancer, a protease inhibitor, or any combination thereof.

54. The pharmaceutical composition of claim 52, wherein the pharmaceutical composition is formulated for oral administration.

55. (canceled)

56. A method of transporting a polypeptide construct from the gastrointestinal tract of a subject in need thereof to the circulatory system of the subject, the method comprising orally administering to the subject the polypeptide construct of claim 1 or claim 30 or the pharmaceutical composition of claim 52.

57. The method of claim 56, wherein the subject is a human.

58. The method of claim 56 or 57, wherein the second polypeptide is separated from the remainder of the polypeptide construct after transport into the circulatory system.

59. The method of claim 58, wherein the second polypeptide comprises an N-terminal or C-terminal adduct selected from A, GA, RGA, GRGA, or a combination thereof.

60-70. (canceled)
